(19) United States
(12) Patent Application Publication
(10) Pub. No.: US 2025/0127191 A1
(43) Pub. Date: Apr. 24, 2025

(54) ANTI-INFLAMMATORY FEED FORMULATION AND USES THEREOF

(71) Applicant: Nat-Phen Nutrition, Varennes (CA)

(72) Inventor: Yves MARCHAND, Varennes (CA)

(21) Appl. No.: 18/730,655

(22) PCT Filed: Jan. 20, 2023
(86) PCT No.: PCT/CA2023/050058

§ 371 Date: Jul. 19, 2024

Related U.S. Application Data
(60) Provisional application No. 63/301,272, filed on Jan. 20, 2022.

(51) Int. Cl. A23K 20/111; A23K 10/30; A23K 20/105; A23K 20/121; A23K 20/142; A23K 20/174; A23K 20/195; A23K 20/20

(52) U.S. Cl. CPC A23K 20/111; A23K 10/30; A23K 20/105; A23K 20/121; A23K 20/142; A23K 20/174; A23K 20/195; A23K 20/20

(57) ABSTRACT

The present document describes a livestock dietary supplement composition which comprises a sialagogue comprising cinnamaldehyde, an anti-inflammatory comprising a salicylate, an expectorant comprising menthol, an antimicrobial comprising carvacrol, anethole and a ginger essential oil; and a sufficient amount of an acceptable carrier. The present document also describes methods and uses of the livestock dietary supplement composition for feeding a livestock animal, and/or for reducing body temperature, systemic inflammation, localized inflammation, or cellular oxidation and/or for improving fertility, and/or for appeasing, and/or for the prevention of body shrinking, and/or for increasing the number of eggs laid by, a livestock animal in need thereof.

### CROSS-REFERENCE TO RELATED APPLICATIONS

This application claims priority of U.S. Provisional Patent Application No. 63/301,272 filed on Jan. 20, 2022, the specification of which is hereby incorporated by reference in its entirety.

### BACKGROUND

### (a) Field

The subject matter disclosed generally relates to livestock dietary supplement compositions. More specifically, the subject matter relates to livestock dietary supplement compositions comprising a sialagogue, comprising cinnamaldehyde, an anti-inflammatory, an expectorant, an antimicrobial, and an acceptable carrier.

### (b) Related Prior Art

Disease or undesirable condition may occur in animal subjects from any one or more of a long list of causes, including microbial infection, exposure to toxins, prolonged exposure to stress, and a diet that is either deficient in one or more essential substances, contains an excess of harmful substances, such as salt, fats, cholesterol, etc., or is otherwise unbalanced. Alternatively, or in addition, the onset and development of disease or undesirable condition may have a genetic component.

Oxidative damage is caused by the action of highly reactive species (such as free radicals or peroxides) on the cells and tissues of the animal body. A free radical is any atom or group of atoms, which has one or more unpaired electrons. Free radicals are uncharged high-energy species that are highly reactive. Peroxides are compounds containing linked pairs of oxygen atoms. Organic peroxides can act as sources of free radicals.

A major source of such reactive species is molecular oxygen, which is converted into a “reactive oxygen species” (ROS). The loss of an electron from molecular oxygen will produce a free radical species, which reacts with cells or tissue in the body causing damage including lipid peroxidation, which leads to cell death. ROS can be produced in the body of an aerobic organism during metabolism, for example by the respiratory burst of phagocytes, mitochondrial oxidative phosphorylation and the xanthine oxidase system. In addition, ROS are produced in the environment, for example by UV light, pollutants, or ionizing radiation.

Exposure of the body to agents causing oxidative damage results in inflammation, auto immune diseases, cancers, muscle disorders, lung inflammation and general ageing. Oxidative damage to different parts of the body causes or exacerbates several disorders. Oxidative stress occurs when the production of reactive oxygen species exceeds the body's natural capacity for removal of the ROS. Systemic and localized inflammation, most of the time, involves reaction of oxidative stresses and production of inflammatory markers, such as protein kinase C, lactic acid, haptoglobin, and cortisol. Other consequences of oxidative stress, body temperature increases and/or systemic inflammation, are the reduction of oxygenation of cells, particularly smooth and red muscle cells, and the loss of equilibrium of the immune and hormonal systems.

Therefore, any new treatment or therapy which may decrease or mitigate stress, inflammation and body temperature may be beneficial, and may improve animal wellbeing and performance.

Therefore, there is a need for dietary supplement compositions that may decrease or mitigate oxidative stress in animals.

### SUMMARY

According to an embodiment, there is provided a livestock dietary supplement composition comprising:
a) from about 0.35% to about 4% w/w of a sialagogue comprising cinnamaldehyde;
b) from about 2% to about 10% w/w of an anti-inflammatory comprising a salicylate;
c) from about 0.1% to about 13% w/w of an expectorant comprising menthol;
d) from about 0.0000022% to about 0.04% w/w of an antimicrobial comprising carvacrol, anethole and a ginger essential oil; and
e) a sufficient amount of an acceptable carrier to make up to 100% w/w.

The salicylate may be methyl salicylate, magnesium salicylate, choline salicylate, bismuth subsalicylate, salsalate, salicin, salicylic acid, acetylsalicylic acid and combinations thereof.

The salicylate may be provided by a wintergreen essential oil.

The expectorant may further comprise menthone and eucalyptol.

The expectorant may comprise a peppermint essential oil.

In the livestock dietary supplement composition of the present invention, in the antimicrobial, the carvacrol may be provided by a thyme essential oil, an oregano essential oil, a pepperwort essential oil, a wild bergamot essential oil, a Satureja essential oil, a marjoram essential oil, a Origanum Dictamnus essential oil, and combinations thereof.

In the antimicrobial, the anethole may be provided by an anise essential oil, a liquorice, a liquorice extract, and combinations thereof.

The livestock dietary supplement composition may further comprise from about 2% to about 8% w/w of an astringent comprising an edible polyphenolic compound.

The edible polyphenolic compound may be a tannin.

The livestock dietary supplement composition may further comprise from about 0,00001 to about 0,00005% w/w of a chromium.

The chromium may be chromium picolinate, an amino acid chelate of chromium, or combinations thereof.

The livestock dietary supplement composition may further comprise a vitamin.

The vitamin may be vitamin C, vitamin D, vitamin E, folic acid, or combinations thereof.

The anti-inflammatory may further comprise a coumarin.

The livestock dietary supplement composition may further comprise a thickening agent.

The thickening agent may be locust bean gum.

The acceptable carrier may comprise a clay, an aluminosilicate, and combinations thereof.

The livestock dietary supplement composition may comprise:
a) from about 0.9% to about 1.2% w/w of cinnamaldehyde;
b) from about 2% to about 3% w/w of a wintergreen oil;
c) from about 0.1% to about 0.15% w/w of a peppermint oil;
d) from about 0.06% to about 0.085% w/w of an antimicrobial comprising carvacrol, anethole, a ginger essential oil, and liquorice; and
e) an acceptable carrier.

According to another embodiment, there is provided a livestock feed composition comprising the livestock dietary supplement composition of the present invention.

According to another embodiment, there is provided a method of feeding livestock comprising administering an effective amount of the livestock dietary supplement composition of the present invention, or the livestock feed composition of the present invention, to a livestock animal in need thereof.

According to another embodiment, there is provided a method of reducing body temperature, systemic inflammation, localized inflammation, or cellular oxidation, of a livestock animal comprising administering an effective amount of the livestock dietary supplement composition of any one of the present invention, or the livestock feed composition of the present invention, to the livestock animal in need thereof.

According to another embodiment, there is provided a method of improving fertility of a livestock animal comprising administering an effective amount of the livestock dietary supplement composition of the present invention, or the livestock feed composition of the present invention, to the livestock animal in need thereof.

According to another embodiment, there is provided a method of increasing the growth rate of a livestock animal comprising administering an effective amount of the livestock dietary supplement composition of the present invention, or the livestock feed composition of the present invention, to the livestock animal in need thereof.

According to another embodiment, there is provided a method of appeasing a livestock animal comprising administering an effective amount of the livestock dietary supplement composition of the present invention, or the livestock feed composition of the present invention, to the livestock animal in need thereof.

According to another embodiment, there is provided a method for the prevention of body shrinking of a livestock animal comprising administering an effective amount of the livestock dietary supplement composition of the present invention, or the livestock feed composition of the present invention, to the livestock animal in need thereof.

According to another embodiment, there is provided a method of increasing the number of eggs laid by a bird comprising administering an effective amount of the livestock dietary supplement composition of the present invention, or the livestock feed composition of the present invention, to the bird in need thereof.

According to another embodiment, there is provided a use of the livestock dietary supplement composition of the present invention, or the livestock feed composition of the present invention, for feeding a livestock animal in need thereof.

According to another embodiment, there is provided a use of the livestock dietary supplement composition of the present invention, or the livestock feed composition of the present invention, for reducing body temperature, systemic inflammation, localized inflammation, or cellular oxidation, of a livestock animal in need thereof.

According to another embodiment, there is provided a use of the livestock dietary supplement composition of the present invention, or the livestock feed composition of the present invention, for improving fertility of a livestock animal in need thereof.

According to another embodiment, there is provided a use of the livestock dietary supplement composition of the present invention, or the livestock feed composition of the present invention, for increasing the growth rate of a livestock animal in need thereof.

According to another embodiment, there is provided a use of the livestock dietary supplement composition of the present invention, or the livestock feed composition of the present invention, for appeasing a livestock animal in need thereof.

According to another embodiment, there is provided a use of the livestock dietary supplement composition of the present invention, or the livestock feed composition of the present invention, for the prevention of body shrinking of a livestock animal in need thereof.

According to another embodiment, there is provided a use of the livestock dietary supplement composition of the present invention, or the livestock feed composition of the present invention, for increasing the number of eggs laid by a bird in need thereof.

According to another embodiment, there is provided a livestock dietary supplement composition of the present invention, or the livestock feed composition of the present invention, for use in feeding a livestock animal in need thereof.

According to another embodiment, there is provided a livestock dietary supplement composition of the present invention, or the livestock feed composition of the present invention, for use in reducing body temperature, systemic inflammation, localized inflammation, or cellular oxidation, of livestock animal in need thereof.

According to another embodiment, there is provided a livestock dietary supplement composition of the present invention, or the livestock feed composition of the present invention, for use in improving fertility of a livestock animal in need thereof.

According to another embodiment, there is provided a livestock dietary supplement composition of the present invention, or the livestock feed composition of the present invention, for use in increasing the growth rate of a livestock animal in need thereof.

According to another embodiment, there is provided a livestock dietary supplement composition of the present invention, or the livestock feed composition of the present invention, for use in appeasing a livestock animal in need thereof.

According to another embodiment, there is provided a livestock dietary supplement composition of the present invention, or the livestock feed composition of the present invention, for use in the prevention of body shrinking of a livestock animal in need thereof.

According to another embodiment, there is provided a livestock dietary supplement composition of the present invention, or the livestock feed composition of the present invention, for use in increasing the number of eggs laid by a bird in need thereof.

The following terms are defined below.

The term “dietary supplement” is intended to mean a manufacture product intended to supplement the diet of a subject. The supplement may be a pill, capsule, tablet, powder, granules or liquid. A dietary supplement can provide nutrients either extracted from food sources or that are synthetic in order to increase the quantity of their consumption. Dietary supplements can also contain substances that have not been confirmed as being essential to life but are marketed as having a beneficial biological effect.

The term “food” is intended to mean any substance consumed to provide nutritional support for an organism or subject. Food is usually of plant, animal or fungal origin, and contains essential nutrients, such as carbohydrates, fats, proteins, vitamins, or minerals.

The term “sialogogue” is intended to mean a drug or substance that increases the production of saliva.

The term “anti-inflammatory” is intended to mean a substance or treatment that reduces inflammation or swelling. Anti-inflammatory drugs, also called anti-inflammatories, antiphlogistics or even deflammatories, make up about half of analgesics. These drugs or compounds remedy pain by reducing inflammation.

The term “expectorant” is intended to mean a substance or compound which promotes the secretion of sputum (the mixture of saliva and mucus) by the air passages, used to treat coughs, for example.

The term “antimicrobial” is intended to mean an agent (a compound, or mixture of compounds) that kills microorganisms (a microbicide) or stops their growth (a microbiostatic). An antimicrobial can be grouped according to the microorganisms they act primarily against. For example, antibiotics, bactericides or bacteriostatics are used against bacteria, and antifungals are used against fungi. Antimicrobials may have efficacy against one or more type of microbes.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition or other compositions in general, is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions or other compositions in general of the present invention encompass any composition made by admixing a compound of the present invention and a pharmaceutically acceptable carrier. By “pharmaceutically acceptable” or “acceptable” it is meant the carrier, diluent or excipient must be compatible with the other ingredients of the formulation and not deleterious to the recipient thereof. According to some embodiments, the composition may be an ophthalmic composition, which may be formulated as any one of a solution, an ointment, a cream, a lotion, eye drops or an eye gel.

The term “subject” as used herein, is a human patient or other animal such as another mammal including a bovine, an ovine, a canine, a caprine, an equine, a feline, a porcine, other primates and a rodent. Birds may also be included, for example chicken, turkey or duck.

The term “osteoarthritis” relates to a degenerative disease of the joint that causes joint pain and stiffness. The subject, such as animal, express permanent discomfort and demonstrate chronic pain that limit all physical movement of the animal and therefore end up in strong inflammation and rapid degeneration.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1 illustrates a graph of the distribution of parities within two iso-parity experimental groups of Holstein-breed dairy cows fed with dietary supplement composition of the present invention until calving (trial group) or not (control group) as described in Example 4.

FIG. 2 illustrates graphs of daily individual average of milk (in kg milk/day), fat (in kg fat/day) and protein (in kg proteins/day) production and milk conductivity (in Sm/cm) during the first 5 months of lactation of Holstein-breed dairy cows fed with dietary supplement composition of the present invention until calving (trial group) or not (control group) as described in Example 4.

FIG. 3 illustrates 5-month means and standard deviations of daily individual average of milk (in kg), fat (in kg) and protein (in kg) production, milk conductivity (in Sm/cm) and number of inseminations to obtain a gestation in cows fed with dietary supplement composition of the present invention until calving (trial group) or not (control group) as described in Example 4.

FIG. 4 illustrates means and standard deviations of daily individual average of milk production (in kg) and milk conductivity based on 5 months period per fortnight in cows fed with dietary supplement composition of the present invention until calving (trial group) or not (control group) as described in Example 4.

FIG. 5 illustrates means and standard deviations of daily individual average of fat (in kg) and protein (in kg) production based on 5 months period per fortnight in cows fed with dietary supplement composition of the present invention until calving (trial group) or not (control group) as described in Example 4.

FIG. 6 illustrates graphs of percentage of change in monthly mean milk conductivity and monthly standard deviations conductivity based on 5 months period per fortnight for cows fed with dietary supplement composition of the present invention until calving or not as described in Example 4.

FIG. 7 describes joint paint symptoms in dogs and associated behaviors.

FIG. 8 illustrates a schematic drawing of the potential therapeutic approach to inhibit the progression of oxidative activity by dietary supplement composition of the present invention.

FIG. 9 illustrates a graph of the response speed in days in dogs showing signs of osteoarthritis fed with a dietary supplement composition of the present invention as described in Example 5.

FIG. 10 illustrates a graph of the response in dogs showing signs of osteoarthritis fed with a dietary supplement composition of the present invention as described in Example 5.

It will be noted that throughout the appended drawings, like features are identified by like reference numerals.

### DETAILED DESCRIPTION

In embodiments there is disclosed a livestock dietary supplement composition which comprises a sialagogue, an anti-inflammatory, an expectorant, an antimicrobial, a sufficient amount of an acceptable carrier to make up to 100% w/w.

For the purposes of this invention the components or compounds may be provided from natural sources or from synthetic sources. The components or compounds may be provided as isolated compounds of natural or of synthetic origin. The components may be also provided from extracts, for examples as extracts from plants. Plant extract may be dried or partially dried, in the form of a powder, in oil, liquid (either as a solution or as an oil), crushed, an extract, fresh or semi-solid. The component or compound can be extracted and partially or fully purified from the plant. The components or compounds may can be provided by one plant/herb or by a mixture of plants or herbs, for example, one or more of wintergreen, rosemary, nutmeg, oregano, basil and coriander. For the purposes of this invention, any part of herb or plant can be used (for example, leaf, stem, bark, bulb, root, fruit, flower, or seed). The herb material can be dried, semi-dried, fresh, crushed, in oil, in solution as a powder, liquid (as a solution or as an oil) or semi-solid. Preferred forms of dried plan extract include those containing between 0 and 40% w/w water or between 0 and 10% w/w or between 0 and 5% w/w.

### Sialagogue

As used herein, the term “sialagogue” is intended to mean a drug or a substance that increases the production of saliva. In embodiments, the sialagogue stimulates any functioning salivary gland tissue to produce more saliva. Saliva has a bactericidal effect, and it is believed that fungal infections such as oral candidosis also can be decreased by the increased flow rate of saliva.

In embodiments, the sialagogue compound used in the present invention comprises cinnamaldehyde. Cinnamaldehyde is an organic compound that may be obtained from the bark of cinnamon trees, or other species of the genus Cinnamomum. In embodiment, cinnamaldehyde may be obtained from any suitable source, synthetic or natural. For example, a natural source of this compound is the essential oil of cinnamon, particularly cinnamon bark, which may comprise up to about 90% cinnamaldehyde.

Other sialagogues include but are not limited to aloe vera or extracts thereof, ginger, or ginger extracts, such as ginger essential oil, marshmallow root (Malva spp.), or extracts thereof, Hollyhock root (Alcea spp.), or extracts thereof, Cactus nopal (Opuntia spp.) or extracts thereof, Spilanthes (Spilanthes acmella), or extracts thereof, sweet pepper (Capsicum annuum) or extracts thereof, and xylitol.

In embodiments, the sialagogue may represent from about 0.35% to about 0.5% w/w, or from about 0.35% to about 1% w/w, or from about 0.35% to about 2% w/w, or from about 0.35% to about 3% w/w, or from about 0.35% to about 4% w/w, or from about 0.5% to about 1% w/w, or from about 0.5% to about 2% w/w, or from about 0.5% to about 3% w/w, or from about 0.5% to about 4% w/w, or from about 1% to about 2% w/w, or from about 1% to about 3% w/w, or from about 1% to about 4% w/w, or from about 2% to about 3% w/w, or from about 3% to about 4% w/w, or from about 0.9% to about 1.5% w/w, or from about 0.9% to about 1.2% w/w, or about 0.35%, 0.5%, 0.9%, 1%, 1.2%, 1.5%, 2%, 3%, 4% of the total weight of the composition of the present invention.

### Anti-Inflammatory

As used herein, the term “anti-inflammatory” is intended to mean a substance or treatment that reduces inflammation or swelling. Anti-inflammatory drugs, also called anti-inflammatories, antiphlogistics or even deflammatories, make up about half of analgesics. These drugs or compounds remedy pain by reducing inflammation.

In embodiments, the anti-inflammatory used in the present invention comprises a salicylate. Salicylic acid, salts thereof and esters thereof are known as salicylates, and they are organic compounds that are precursors to, and metabolites of acetylsalicylic acid, well known for its anti-inflammatory properties. Salicylates and salicylic acid are used in the production of other pharmaceuticals, and they have long been key starting materials for making acetylsalicylic acid. In embodiments, the salicylate may be any one of methyl salicylate, magnesium salicylate, choline salicylate, bismuth subsalicylate, salsalate, salicin, salicylic acid, acetylsalicylic acid and combinations thereof.

In embodiment, the salicylate may be provided by any suitable source of salicylate. For example, and according to an embodiment, the salicylate may be provided by wintergreen essential oil. Wintergreen essential oil may be extracted from Gaultheria species, that share the common characteristic of producing oil of wintergreen, also known as wintergreen essential oil. Wintergreen essential oil is a pale yellow or pinkish fluid liquid that is strongly aromatic with a sweet, woody odor. It comprises as an active ingredient methyl salicylate (about as much as 98% w/w), α-pinene, myrcene, delta-3-carene, limonene, 3,7-guaiadiene, and delta-cadinene, that give such plants a distinctive “medicinal” smell.

According to another embodiment, the anti-inflammatory may further comprise coumarin. Coumarin is a colorless crystalline solid with a sweet odor resembling the scent of vanilla and a bitter taste. It is found in many plants, where it may serve as a chemical defense against predators, and there is evidence that it has anti-inflammatory properties.

In embodiments, the anti-inflammatory may represent from about 2% to about 3% w/w, or from about 2% to about 4% w/w, or from about 2% to about 5% w/w, or from about 2% to about 6% w/w, or from about 2% to about 7% w/w, or from about 2% to about 8% w/w, or from about 2% to about 9% w/w, or from about 2% to about 10% w/w, or from about 2.25% to about 3% w/w, or from about 2.25% to about 4% w/w, or from about 2.25% to about 5% w/w, or from about 2.25% to about 6% w/w, or from about 2.25% to about 7% w/w, or from about 2.25% to about 8% w/w, or from about 2.25% to about 9% w/w, or from about 2.25% to about 10% w/w, or from about 2.5% to about 3% w/w, or from about 2.5% to about 4% w/w, or from about 2.5% to about 5% w/w, or from about 2.5% to about 6% w/w, or from about 2.5% to about 7% w/w, or from about 2.5% to about 8% w/w, or from about 2.5% to about 9% w/w, or from about 2.5% to about 10% w/w, or from about 2.75% to about 3% w/w, or from about 2.75% to about 4% w/w, or from about 2.75% to about 5% w/w, or from about 2.75% to about 6% w/w, or from about 2.75% to about 7% w/w, or from about 2.75% to about 8% w/w, or from about 2.75% to about 9% w/w, or from about 2.75% to about 10% w/w, or from about 3% to about 4% w/w, or from about 3% to about 5% w/w, or from about 3% to about 6% w/w, or from about 3% to about 7% w/w, or from about 3% to about 8% w/w, or from about 3% to about 9% w/w, or from about 3% to about 10% w/w, or from about 4% to about 5% w/w, or from about 4% to about 6% w/w, or from about 4% to about 7% w/w, or from about 4% to about 8% w/w, or from about 4% to about 9% w/w, or from about 4% to about 10% w/w, or from about 5% to about 6% w/w, or from about 5% to about 7% w/w, or from about 5% to about 8% w/w, or from about 5% to about 9% w/w, or from about 5% to about 10% w/w, or from about 6% to about 7% w/w, or from about 6% to about 8% w/w, or from about 6% to about 9% w/w, or from about 6% to about 10% w/w, or from about 7% to about 8% w/w, or from about 7% to about 9% w/w, or from about 7% to about 10% w/w, or from about 8% to about 9% w/w, or from about 8% to about 10% w/w, or from about 9% to about 10% w/w, or about 2%, 2.25%, 2.5%, 2.75%, 3%, 4%, 5%, 6%, 7%, 8%, 9%, or 10% of the total weight of the composition of the present invention.

### Expectorant

As used herein, the term “expectorant” refers to substances or compounds which promotes the secretion of sputum (the mixture of saliva and mucus) by the air passages, used to treat coughs, for example. Expectorants are a class of mucoactive agents that aid in the clearance of mucus from the upper and lower airways, including the lungs, bronchi, and trachea. Expectorants are believed to increase airway water or the volume of airway secretions.

In embodiments, the expectorant used in the present invention comprises menthol. Menthol is an organic compound, more specifically a monoterpenoid, made synthetically or obtained from the oils of corn mint, peppermint, or other mints. It is a waxy, crystalline substance, clear or white in color, which is solid at room temperature and melts slightly above. The main form of menthol occurring in nature is (−)-menthol, which is assigned the (1R,2S,5R) configuration.

In embodiments, the expectorant may also comprise menthone, which also is a monoterpene with a minty flavor that occurs naturally in a number of essential oils, particularly mint essential oils. I-Menthone (or (2S,5R)-trans-2-isopropyl-5-methylcyclohexanone), is the most abundant in nature of the four possible stereoisomers of menthone. It is structurally related to menthol, which has a secondary alcohol in place of the carbonyl. Menthone is used in flavoring, perfume and cosmetics for its characteristic aromatic and minty odor.

In embodiments, the expectorant may also comprise eucalyptol, which is a monoterpenoid. It is a colorless liquid, and a bicyclic ether. Eucalyptol also has a fresh mint-like smell and a spicy, cooling taste. It is insoluble in water, but miscible with organic solvents. Eucalyptol makes up 90% w/w of eucalyptus oil.

In embodiments, the expectorant may be provided from any suitable source of the compounds, be they synthetic or natural sources. For example, menthol and menthone may be provided from mint essential oils, particularly from peppermint essential oils. According to another embodiment, eucalyptol may be provided from eucalyptus essential oils, and peppermint oils.

In embodiments, the expectorant may represent from about 0.1% to about 0.15% w/w, or from about 0.1% to about 0.5% w/w, or from about 0.1% to about 1% w/w, or from about 0.1% to about 2% w/w, or from about 0.1% to about 3% w/w, or from about 0.1% to about 4% w/w, or from about 0.1% to about 5% w/w, or from about 0.1% to about 6% w/w, or from about 0.1% to about 7% w/w, or from about 0.1% to about 8% w/w, or from about 0.1% to about 9% w/w, or from about 0.1% to about 10% w/w, or from about 0.1% to about 11% w/w, or from about 0.1% to about 12% w/w, or from about 0.1% to about 13% w/w, or from about 0.15% to about 0.5% w/w, or from about 0.15% to about 1% w/w, or from about 0.15% to about 2% w/w, or from about 0.15% to about 3% w/w, or from about 0.15% to about 4% w/w, or from about 0.15% to about 5% w/w, or from about 0.15% to about 6% w/w, or from about 0.15% to about 7% w/w, or from about 0.15% to about 8% w/w, or from about 0.15% to about 9% w/w, or from about 0.15% to about 10% w/w, or from about 0.15% to about 11% w/w, or from about 0.15% to about 12% w/w, or from about 0.15% to about 13% w/w, or from about 0.5% to about 1% w/w, or from about 0.5% to about 2% w/w, or from about 0.5% to about 3% w/w, or from about 0.5% to about 4% w/w, or from about 0.5% to about 5% w/w, or from about 0.5% to about 6% w/w, or from about 0.5% to about 7% w/w, or from about 0.5% to about 8% w/w, or from about 0.5% to about 9% w/w, or from about 0.5% to about 10% w/w, or from about 0.5% to about 11% w/w, or from about 0.5% to about 12% w/w, or from about 0.5% to about 13% w/w, or from about 1% to about 2% w/w, or from about 1% to about 3% w/w, or from about 1% to about 4% w/w, or from about 1% to about 5% w/w, or from about 1% to about 6% w/w, or from about 1% to about 7% w/w, or from about 1% to about 8% w/w, or from about 1% to about 9% w/w, or from about 1% to about 10% w/w, or from about 1% to about 11% w/w, or from about 1% to about 12% w/w, or from about 1% to about 13% w/w, or from about 2% to about 3% w/w, or from about 2% to about 4% w/w, or from about 2% to about 5% w/w, or from about 2% to about 6% w/w, or from about 2% to about 7% w/w, or from about 2% to about 8% w/w, or from about 2% to about 9% w/w, or from about 2% to about 10% w/w, or from about 2% to about 11% w/w, or from about 2% to about 12% w/w, or from about 2% to about 13% w/w, or from about 3% to about 4% w/w, or from about 3% to about 5% w/w, or from about 3% to about 6% w/w, or from about 3% to about 7% w/w, or from about 3% to about 8% w/w, or from about 3% to about 9% w/w, or from about 3% to about 10% w/w, or from about 3% to about 11% w/w, or from about 3% to about 12% w/w, or from about 3% to about 13% w/w, or from about 4% to about 5% w/w, or from about 4% to about 6% w/w, or from about 4% to about 7% w/w, or from about 4% to about 8% w/w, or from about 4% to about 9% w/w, or from about 4% to about 10% w/w, or from about 4% to about 11% w/w, or from about 4% to about 12% w/w, or from about 4% to about 13% w/w, or from about 5% to about 6% w/w, or from about 5% to about 7% w/w, or from about 5% to about 8% w/w, or from about 5% to about 9% w/w, or from about 5% to about 10% w/w, or from about 5% to about 11% w/w, or from about 5% to about 12% w/w, or from about 5% to about 13% w/w, or from about 6% to about 7% w/w, or from about 6% to about 8% w/w, or from about 6% to about 9% w/w, or from about 6% to about 10% w/w, or from about 6% to about 11% w/w, or from about 6% to about 12% w/w, or from about 6% to about 13% w/w, or from about 7% to about 8% w/w, or from about 7% to about 9% w/w, or from about 7% to about 10% w/w, or from about 7% to about 11% w/w, or from about 7% to about 12% w/w, or from about 7% to about 13% w/w, or from about 8% to about 9% w/w, or from about 8% to about 10% w/w, or from about 8% to about 11% w/w, or from about 8% to about 12% w/w, or from about 8% to about 13% w/w, or from about 9% to about 10% w/w, or from about 9% to about 11% w/w, or from about 9% to about 12% w/w, or from about 9% to about 13% w/w, or from about 10% to about 11% w/w, or from about 10% to about 12% w/w, or from about 10% to about 13% w/w, or from about 11% to about 12% w/w, or from about 11% to about 13% w/w, or from about 12% to about 13% w/w, 0.1%, 0.15%, 0.5% 1%, 2%, 3%, 4%, 5%, 6%, 7%, 8%, 9%, 10%, 11%, 12%, 13% of the total weight of the composition of the present invention.

### Antimicrobial

As used herein, “antimicrobial” refers to agents (a compound, or mixture of compounds) that kill microorganisms (a microbicide) or stops their growth (a microbiostatic). An antimicrobial can be grouped according to the microorganisms they act primarily against. For example, antibiotics, bactericides or bacteriostatics are used against bacteria, and antifungals are used against fungi. Antimicrobials may have efficacy against one or more type of microbes.

According to an embodiment, the antimicrobial used in the composition of the present invention comprises a mixture of carvacrol, anethole and a ginger essential oil. In embodiments, the antimicrobial may be provided from any suitable source of the compounds, be they synthetic or natural sources.

Carvacrol is a monoterpenoid phenol. It has a characteristic pungent, warm odor of oregano. Carvacrol is present in the essential oil of Origanum vulgare (oregano), oil of thyme, oil obtained from pepperwort, and wild bergamot. The essential oil of thyme subspecies contains between 5% and 75% w/w of carvacrol, while Satureja (savory) subspecies have a content between 1% and 45% w/w. Origanum majorana (marjoram) and Dittany of Crete are rich in carvacrol, 50% and 60-80% w/w respectively.

In embodiments, in the antimicrobial, the carvacrol may be provided by a thyme essential oil, an oregano essential oil, a pepperwort essential oil, a wild bergamot essential oil, a Satureja essential oil, a marjoram essential oil, a Origanum Dictamnus essential oil, and combinations thereof.

Anethole (also known as anise camphor) is an organic compound that is widely used as a flavoring substance. It is a derivative of phenylpropene, a type of aromatic compound that occurs widely in nature, in essential oils. It is in the class of phenylpropanoid organic compounds. It contributes a large component of the odor and flavor of anise and fennel (both in the botanical family Apiaceae), anise myrtle (Myrtaceae), liquorice (Fabaceae), magnolia blossoms, and star anise (Schisandraceae). It is a colorless, fragrant, mildly volatile liquid. Anethole is only slightly soluble in water but exhibits high solubility in ethanol. This trait causes certain anise-flavored liqueurs to become opaque when diluted with water, the ouzo effect.

In embodiments, in the antimicrobial, the anethole may be provided by an anise essential oil, a liquorice, a liquorice extract, and combinations thereof. Liquorice is used for food additives and foods in Japan. In the US, licorice was registered as a GRAS (Generally Recognized As Safe) food by FDA in 1985, approved as a cancer preventive food in the Designer Foods 1990, and ingestion thereof was recommended. In embodiments, glycyrrhizin (glycyrrhizic acid) which is the main sweet-tasting constituent of liquorice root or extracts thereof, and it is 30 to 50 times as sweet as sucrose. Glycyrrhizin is converted into its aglycone by intestinal flora. Flavonoid aglycones are very bioavailable.

In embodiments, the antimicrobial may represent from about 0.0000022% to about 0.002% w/w, or from about 0.0000022% to about 0.004% w/w, or from about 0.0000022% to about 0.005% w/w, or from about 0.0000022% to about 0.006% w/w, or from about 0.002% to about 0.004% w/w, or from about 0.002% to about 0.006% w/w, or from about 0.002% to about 0.006% w/w, or from about 0.004% to about 0.005% w/w, or from about 0.004% to about 0.006% w/w, or from about 0.004% to about 0.005% w/w, or from about 0.005% to about 0.006% w/w, or about 0.0000022%, 0.002%, 0.004%, 0.006% of the total weight of the composition of the present invention.

### Astringents

According to another embodiment, the livestock dietary supplement composition of the present invention may further comprise an astringent. As used herein, the term astringent is intended to refer to a taste sensation that puckers the mouth, numbs the tongue, and constricts the throat, and is also associated with pungent, tingly, and drying sensations in the mouth and tongue. This taste is caused by astringents such as tannins. The astringents are believed to absorbs excess moisture, cleanses the mucus membranes, scrapes fat, improves absorption. Unlike other tastes, astringency is not perceived via taste receptors but is a tactile sensation caused by the aggregation of saliva proteins. For example, the astringent taste is in unripened bananas, unripe persimmons and cashew fruits; and acorns dominantly, which prevents them from being eaten.

According to an embodiment, the astringent may be an edible polyphenolic compound. Edible polyphenols, or edible polyphenol antioxidant are a hypothetical type of antioxidants that contain a polyphenolic substructure. There are over 4,000 distinct species polyphenol antioxidants, mostly from plants. These polyphenols antioxidant may have antioxidant activity in vitro. They are hypothesized to affect cell-to-cell signaling, receptor sensitivity, inflammatory enzyme activity or gene regulation. In an embodiment, the edible polyphenolic compound may be a tannin. Tannins are astringent, polyphenolic biomolecules that bind to and precipitate proteins and various other organic compounds including amino acids and alkaloids. Tannins have molecular weights ranging from 500 to over 3,000 (gallic acid esters) and up to 20,000 Daltons (proanthocyanidins).

Other polyphenol that are edible and may be included in the present invention include genistein, daidzein, quercetin, rutin, catechin, epigallocatechin gallate, hesperidin, nobiletin, tyrosol, hydroxytyrosol, oleuropein, naringenin, caffeic acid, apple polyphenol, tea polyphenol, gallic acid and the like. Preferred are genistein, daidzein, quercetin, rutin, catechin, epigallocatechin gallate, hesperidin, nobiletin, naringenin, apple polyphenol and tea polyphenol. These polyphenols may be used alone or in a mixture of two or more kinds thereof. Genistein here is polyphenol contained in leguminous plants such as soybean and the like.

In embodiments, the astringent may represent about 2% to about 8% w/w, or from about 1% to about 4% w/w of the total weight of the composition of the present invention.

According to an embodiment, the livestock dietary supplement composition of the present invention may comprise from about 0.9% to about 1.2% w/w, or about 0.9% to about 1.1% w/w, or about 0.9% to about 1.0% w/w, or about 1% to about 1.2% w/w, or about 1% to about 1.1% w/w, or about 1.1% to about 1.2% w/w, or about 0.9%, 1.0%, 1.1%, 1.2% of cinnamaldehyde; from about 2% to about 3% w/w, or from about 2.5% to about 3% w/w, or from about 2% to about 2.5% w/w of a wintergreen oil; from about 0.1% to about 0.15% w/w, or about 0.1%, or 0.15% w/w of a peppermint oil; from about 0.06% to about 0.085% w/w, or from about 0.06% to about 0.075% w/w, or from about 0.06% to about 0.07% w/w, or about 0.06%, 0.07%, 0.75%, 0.85% w/w of an antimicrobial comprising carvacrol, anethole, a ginger essential oil, and liquorice; a sufficient amount of an acceptable carrier to make up to 100% w/w.

### Other Ingredients

According to another embodiment, the livestock dietary supplement composition of the present invention may further comprise a number of additional ingredients.

According to an embodiment, the livestock dietary supplement composition may comprise a chromium, such as for example chromium picolinate, an amino acid chelate of chromium, or combinations thereof. Chromium is an essential trace mineral that can improve insulin sensitivity and enhance protein, carbohydrate, and lipid metabolism. It is a metallic element that is needed very small quantities. Chromium may be present from about 0,00001 to about 0,00005% w/w of the total weight of the composition of the present invention.

According to an embodiment, the livestock dietary supplement composition may comprise a vitamin. The vitamin may be an antioxidant vitamin, or another vitamin, such as for example, vitamin D, or a vitamin B (group) (e.g., vitamins B1, B2, B6, B12, folic acid, niacin, biotin, pantothenic acid and the like). In embodiments, the vitamin is vitamin C (L-ascorbic acid), fat-soluble vitamins (e.g., vitamin A, vitamin D, vitamin E, vitamin K) folic acid, or combinations thereof.

The vitamins of the invention are compounds which can inactivate free radical species or the sources of free radicals including reactive oxygen species and hydrogen peroxide. Examples of such antioxidant vitamins include vitamin C, vitamin E and beta-carotene.

Vitamin C is a water-soluble substance. Vitamin C has several important roles in the body. It has an essential role in the maintenance of healthy teeth, gums and bones. It aids the healing of wounds, scar tissue and fractures and strengthens blood vessels. Vitamin C also builds resistance to infection and aids in the prevention and treatment of the common cold. Vitamin C is also one of the major antioxidant nutrients.

The food supplement of the invention will optionally contain vitamin C at a level from 10 mg to 1 gram per kilogram body weight per day. The vitamin C may be in any form. It may be liquid, semi-solid or solid.

Vitamin E is a collective term for several biologically similar compounds, including those called tocopherols and tocotrienols, which share the same biological activity. The selenium containing enzyme glutathione peroxidase together with vitamin E helps to protect cells against free radical induced damage. Vitamin E acts as a scavenger of free radicals. Vitamin C may assist by reducing the tocopheroxyl radicals formed by the scavenging. In addition, vitamin E helps to block lipid peroxidation and may also form an important part of the membrane structure due to its interaction with membrane phospholipids. It has also been suggested that Vitamin E plays an important role in the functioning of the immune system. The most biologically active biological form of vitamin E in animal tissue is α-tocopherol. Vitamin E cannot be synthesised in vivo. Forms of vitamin E for the present invention include D-α-tocopherol, D-α-tocopherol acetate, DL-α-tocopherol and DL-alpha-tocopherol acetate.

Units of vitamin E can be expressed as International Units (IU), where 1 IU of alpha-tocopherol approximates to 1 mg of alpha-tocopherol. Other vitamin E compounds have their IU determined by their biopotency in comparison to alpha-tocopherol as described in McDowell, L. R (1989) Vitamin E: In vitamins in Animal Nutrition, Chapter 4, page 96, Academic Press, UK. Vitamin E is a major antioxidant nutrient and acts in the body as a free radical scavenger. Alpha-tocopherol is the most active antioxidant biological form of vitamin E.

The livestock dietary supplement of the present invention may contain vitamin E at a level from 1 IU to 100 IU per kilogram body weight per day. A further useful point in relation to the use of vitamin E in combination with vitamin C is their potential to act synergistically, and more again when in combination with anti-inflammatory plant extract, such as these containing polyphenolic acids. This may be assisted by the fact that vitamin E is lipid soluble and vitamin C is water-soluble. Alpha-tocopherol is known to sit in the lipid membrane. Ascorbate and α-tocopherol, for example, interact at the interface between cell membranes or lipoproteins and water. Ascorbic acid rapidly reduces α-tocopherol radicals in membranes to regenerate alpha-tocopherol.

In addition, examples of the vitamin-like component include, for example, choline, inositol, p-aminobenzoic acid, pangamic acid, vitamin U, nicotinic acid amide, ester derivatives of vitamins C and E and the like, and further, coenzymes such as FAD, NADH, NADPH and the like, phosphocreatine, ATP and the like.

The composition of the present invention may further contain amino acid or amino acid derivative. Examples of the amino acid and amino acid derivative include alanine, valine, leucine, isoleucine, glycine, proline, serine, glutamine, glutamic acid, arginine, lysine, asparagine, aspartic acid, citrulline, ornithine, carnitine and derivatives thereof. These amino acids may be any of L form, D form and a mixture thereof (e.g., racemate).

In embodiments, the vitamin may represent about 1% to about 8% (w/w), or from about 2% to about 8% (w/w), or from about 3% to about 8% (w/w), or from about 4% to about 8% (w/w), or from about 5% to about 8% (w/w), or from about 6% to about 8% (w/w), or from about 7% to about 8% (w/w), or from about 1% to about 7% (w/w), or from about 2% to about 7% (w/w), or from about 3% to about 7% (w/w), or from about 4% to about 7% (w/w), or from about 5% to about 7% (w/w), or from about 6% to about 7% (w/w), or from about 1% to about 6% (w/w), or from about 2% to about 6% (w/w), or from about 3% to about 6% (w/w), or from about 4% to about 6% (w/w), or from about 5% to about 6% (w/w), or from about 1% to about 5% (w/w), or from about 2% to about 5% (w/w), or from about 3% to about 5% (w/w), or from about 4% to about 5% (w/w), or from about 1% to about 4% (w/w), or from about 2% to about 4% (w/w), or from about 3% to about 4% (w/w), or from about 1% to about 3% (w/w), or from about 2% to about 3% (w/w), or from about 1% to about 2% (w/w), or 1, 2, 3, 4, 5, 6, 7, 8% of the total weight of the composition of the present invention.

### Carriers and Other Formulation Additives

The composition of the present invention may comprise suitable carriers and other formulation additives, to make up the composition of the present invention to 100% w/w. Inert ingredient(s) make up the carrier, as well as any product which results, directly or indirectly, from combination, complexation or aggregation of any two or more of the ingredients, or from dissociation of one or more of the ingredients, or from other types of reactions or interactions of one or more of the ingredients.

A composition of the present invention may further contain other materials such as carriers, disintegrant, lubricant, binder, colorant, anticoagulant, absorption promoter, solubilizing agents of active ingredient, stabilizer, fat and oil, viscosity modifier and the like can be used. In embodiments, the carrier may comprise a clay, an aluminosilicate, a filler and combinations thereof. The filler may be for example grinded corn. In another embodiment, the livestock dietary supplement composition may further comprise a thickening agent, such as for example locust bean gum.

In embodiments, the carrier may be for example, sucrose, lactose, glucose, cornstarch, mannitol, crystalline cellulose, calcium phosphate, calcium sulfate, magnesium sulfate and the like can be mentioned.

In embodiments, the disintegrant may be for example starch, agar, calcium citrate, calcium carbonate, sodium hydrogen carbonate, dextrin, crystalline cellulose, carboxymethylcellulose, tragacanth and the like can be mentioned.

In embodiments, the lubricant may be for example talc, magnesium stearate, polyethylene glycol, silica, hydrogenated vegetable oil and the like can be mentioned. While the above-mentioned binder is not particularly limited, for example, ethyl cellulose, methylcellulose, hydroxypropylmethylcellulose (HPMC), tragacanth, shellac, gelatin, gum arabic, polyvinylpyrrolidone, polyvinyl alcohol, polyacrylic acid, polymethacrylic acid, sorbitol and the like can be mentioned.

In embodiments, the absorption may be for example surfactants such as higher alcohols, higher fatty acids, sucrose fatty acid ester, sorbitan fatty acid ester, sorbitan polyoxyethylene fatty acid ester, polyglycerin fatty acid ester etc., and the like can be mentioned.

In embodiments, the solubilizing agent may be an organic acid such as fumaric acid, succinic acid, malic acid, etc., and the like can be mentioned.

In embodiments, the stabilizer may be for example benzoic acid, sodium benzoate, ethyl parahydroxybenzoate, propylene glycol and the like can be mentioned.

### Livestock Feed Composition and Uses Thereof

In embodiments, there is disclosed a livestock feed composition. Indeed, the livestock dietary supplement composition of the present invention may be incorporated in a livestock feed composition, which comprises feed, as well as the livestock dietary supplement composition.

In use, the livestock dietary supplement composition or the livestock feed composition may be used for multiple purposes.

Administration of the livestock feed composition of the invention to an animal will provide an increased level of compounds with antioxidant activity thus reducing oxidative stress to the body. This will be particularly beneficial where an animal has been, will be, or is subjected to an increase growth, or where an animal is exposed to free radicals due to the environment. The livestock feed composition may help maintaining the health of an animal by supporting or improving the natural defenses of the body.

Controlling the systemic or localized inflammation or body temperature increase in farm animals by giving the animals a livestock feed composition according to the present invention is believed to prevent further damage by environmental management or the use of prescription medication which can be costly and involves the risk of unwanted side effects. Administration of the livestock feed composition of the present invention can reduce the need for prescription medication thereby decreasing the risk of side effects to that medication.

According to another embodiment of the present invention the livestock feed composition may be used in a method for the prophylaxis or treatment of an animal susceptible to or suffering from damages dues to oxidative stress, body temperature increase, systemic and/or localized inflammation, and combinations thereof. The invention also relates to a method of contributing to the prophylaxis or treatment of an animal susceptible to or suffering from an environmental stress effect. In particular the present invention relates to a method of treatment where the stress effect involves an inflammation of the body.

The livestock feed composition of the present invention can be used for the preparation of foods, drinks, functional foods, nutritional supplements, pharmaceutical products, quasi-drugs, cosmetics, animal drugs, pet foods, feeds or prey feeds. As used herein, the term functional foods refers to any food form other than pharmaceutical products, which can be ingested for maintaining health, such as what is referred to as health foods, health supplements, specified health foods, foods with nutrient function claims and the like.

In use, the unit ingestion amount of the livestock dietary supplement composition of the present invention, is from about 1:250 to about 1:4000, or about 1:250 to about 1:3000, or about 1:250 to about 1:2000, or about 1:250 to about 1:1000, or from about 1:500 to about 1:4000, or about 1:500 to about 1:3000, or about 1:500 to about 1:2000, or about 1:500 to about 1:1000, or from about 1:1000 to about 1:4000, or about 1:1000 to about 1:3000, or about 1:1000 to about 1:2000, or from about 1:2000 to about 1:4000, or about 1:2000 to about 1:3000, or from about 1:3000 to about 1:4000, or 1:250, 1:500, 1:1000, 1:2000, 1:3000. 1:4000 (composition: animal feed ratio). The ingestion amount may vary as appropriate depending on the age, body weight, sex, stress level and the like of the animal. In the case of pharmaceutical products, they contain an active ingredient in a one-time ingestion amount, i.e., an amount to be administered by one dose, in a package or bottle for a single consumption and the like. In the case of feed to be provided to animals, the composition of the present invention is provided mixed according to the above ratios directly in the feed, and it is ingested in a continuous manner to the animals. The compositions of the present invention may also be provided to animals, for example females to be inseminated or impregnated, prior to ovulation and during implantation of the embryo. In other embodiments, the compositions of the present invention may also be provided during gestation of the animal. In other embodiments, the compositions of the present invention may be provided to a newly born animal during the first days, weeks or months of life.

According to an embodiment, there is disclosed a method of feeding livestock which comprises administering an effective amount of the livestock dietary supplement composition of the present invention, or the livestock feed composition of the present invention, to a livestock animal in need thereof.

According to an embodiment, there is disclosed a method of reducing body temperature, systemic inflammation, localized inflammation, or cellular oxidation, of a livestock animal comprising administering an effective amount of the livestock dietary supplement composition of the present invention, or the livestock feed composition of the present invention, to a livestock animal in need thereof.

According to an embodiment, there is disclosed a method of improving fertility of a livestock animal comprising administering an effective amount of the livestock dietary supplement composition of the present invention, or the livestock feed composition of the present invention, to a livestock animal in need thereof.

According to an embodiment, there is disclosed a method of increasing the growth rate of a livestock animal comprising administering an effective amount of the livestock dietary supplement composition of the present invention, or the livestock feed composition of the present invention, to a livestock animal in need thereof.

According to an embodiment, there is disclosed a method of appeasing a livestock animal comprising administering an effective amount of the livestock dietary supplement composition of the present invention, or the livestock feed composition of the present invention, to a livestock animal in need thereof.

According to an embodiment, there is disclosed a method for the prevention of body shrinking of a livestock animal comprising administering an effective amount of the livestock dietary supplement composition of the present invention, or the livestock feed composition of the present invention, to a livestock animal in need thereof. As used herein, the expression “body shrinking” is intended to mean the occurrence of quality defects of meat, mechanical skin injuries, and limb fractures in animals, that occur during transport of the animals to the slaughterhouse. This causes the loss of the raw material. This is very disadvantageous for breeders and processing plants, which suffer economic losses.

According to an embodiment, there is disclosed a method of increasing the number of egg laid by a bird comprising administering an effective amount of the livestock dietary supplement composition of the present invention, or the livestock feed composition of the present invention, to a livestock animal in need thereof.

The present invention will be more readily understood by referring to the following examples which are given to illustrate the invention rather than to limit its scope.

### Example 1

### Effects of Polyphenols from Winter Green Oil, Pepper Mint, and Cinnamon as Feed Additive for Pigs Stress Control

Stress is an extremely serious problem of animal production and appears to be a principal element responsible for a majority of production losses. It is a natural reaction of the organism and is an inflammatory mechanism which inhibits other functions of the body to initially make it possible to produce sugar, and then answer the external injuries and insults. New approaches and alternatives to livestock production which make it possible to reduce the problems of the stress would be beneficial. Food is also a means of reducing least helped to control stress. The acquisition of knowledge on the plants with anti-inflammatory properties appears to be an interesting way to support a better metabolic balance. The objective of this example is to investigate the possibility that food may increase the tolerance or resistance to the various stresses in the breeding of pigs.

TABLE 1
Active ingredients

| | | | | |
|---|---|---|---|---|
| | | | | Targeted active |
| Plant name | Format | Origin | Global active material | material |

ANTI-INFLAMMATORY INGREDIENTS

| | | | | |
|---|---|---|---|---|
| Chinese licorice | Powder | Asia | isoflavones, liquiritine, | Ortho acethyl |
| | | | isoliquiry and formoneitine, | salicylate |
| | | | ortho sodium acetyle salicylate | (phenolic |
| | | | | glucosides) |
| Winter green | Oil | Europe and | Methyl salicylate | Methyl salicylate |
| | | Asia | | (Glucosides |
| | | | | phenoliques) |
| Pepper mint | Powder | Europe and | Menthol, phenolic acids | Phenolic acids |
| | | Asia | | |
| Vitamin E | Powder | Asia | Alpha tocopherol | Vitamin E |

BLOOD SUGAR MODULATING INGREDIENTS

| | | | | |
|---|---|---|---|---|
| Chromate | Powder | USA | Chrome | Chrome |
| tripicolinate | | | | |

SIALAGOGUE AND BACTERIOSTATIC INGREDIENTS

| | | | | |
|---|---|---|---|---|
| Cinnamon | Oil | South | Cinnamon aldehyde, eugenol, | Cinnamon |
| | | America, | tanins, coumarin | aldehyde, |
| | | Europe and | | coumarin |
| | | Asia | | |

TABLE 2
Percentages of ingredients

| | | Formula before | Formula A: | Placebo |
|---|---|---|---|---|
| | | substitution | Group A | formula |
| Ingredients | | % w/w | % w/w | % w/w |
| Phytoactive | Methyl salicylate | 0 | 4.0 | None |
| components | of wintergreen oil | | | |
| | Licorice O- | 12.5 | 4.5 | |
| | salicylate | | | |
| | Phenolic acid | 0.5 | 2.5 | |
| | Cinnamaldehyde | 0.5 | 1.0 | |
| | Coumarin | 0.25 | 1.0 | |
| | Tanin | 0.25 | 1.0 | |
| Active | Vitamin E | 64K U.I. or as premix 12.8% | | |
| components | chrome | 200 PPB as premix 10% | | |
| | Tripicolinate | | | |
| | Folic acid | 4000 mg/kg as premix 1% | | |
| Secondary | Argile | Variation of inert ingredients: 10% | | |
| components | Aluminosilicate | | | |
| | Aroma | | | |

| | | |
|---|---|---|
| Ground corn | 76 | 90 |
| Total | 100 | 100 |

Notes:
1. Formulation before substitution: 14% of activity (w/w) of polyphenol of 2 sources, licorice (12.5%) and cinnamon (1.5%).
2. Formulation A: 14% of activity (w/w) of polyphenol of 2 sources, licorice (4.5%), winter green oil (4.0%), mint (2.5%) and cinnamon (3.0%).
3. Coumarines are blood anticoagulants
4. Tanins are astringent polyphenols

### Experimental Protocol

Preparation of the mixture was carried out in several stages. Stage 1 is the preparation of a small sample which was used to evaluate the basic characteristics of the ingredients. (Color, odor, fluidity, density, palatability). The stage 2 was to check how the ingredients could be handled in laboratory production. Stage 3 was the production of test samples to obtain a threshold of acceptability of craving for the animals. This stage required several trials between the farm and the kitchen laboratory. When the threshold of acceptability was reached, a larger sample was produced to tests the products on the animals. The formulas are shown in Table 2 above.

### Pigs and Normal Stress

The project comprises three phases: phase 1 consists in substituting active molecules anti-inflammatory drugs of the liquorice of China (isoflavones, liquiritine, isoliquirie and formoneitine, sodium ortho acetyl salicylate) by other sources coming from the wintergreen oil, and peppermint (methyl salicylate, tannin, menthol, phenolic acid, cinnamon aldehyde). The second phase comprises trying out the mixture on pigs in fattening and normal conditions. The third phase includes adjusting the inert ingredients to improve handling of the product. The formulation is a dietary supplement composition that may be used at a ratio of 1:1000 in complete feed stuff. The effectiveness of the formulation is evaluated by the presence of protein inflammatory in blood, and of the zootechnical results of performances on mortality and the food effectiveness.

### Hypothesis

A dietary supplement composition of the present invention may help control the inflammatory reaction and can have a positive effect on the zootechnical parameters. Also, it may be is possible to substitute sources and different molecules when the substituted sources or molecule has the same biological properties. It is believed that in an animal exposed to dietary supplement composition comprising different ingredients, but having the same function, the response should be the same or be similar. For example, if the anti-inflammatory are effective and they are substituted with other molecules of comparable nature the same biological activity should be observed.

In an example, the ingredient to be substituted is liquorice, which may be substituted with wintergreen essential oil, pepper mint essential oil, and cinnamon. Substitution may help avoid precipitation into the dietary supplement composition.

### Materials & Methods

TABLE 3
Additive dosages according to the sex

| Animals | Quantity |
|---|---|
| Sow | 2 kg/metric ton |
| Boar | 1 kg/metric ton |
| Piglet | 2 kg/metric ton |

TABLE 4
Assessment parameters

| Parameters measured | Polyphenol supplements |
|---|---|
| Biochemistry | |
| a. Haptoglobin (celular oxydation) | Yes |
| b. CPK: (muscle degenerescence) | Yes |
| c. Lactate: (stress time) | Yes |
| Zootechnical: | |
| a. Daily gain | No |
| b. Feed intake | Yes |
| c. Water intake | Yes |
| d. Fertility | Yes |
| Separation-covering time lag | Yes |
| Insemination rate | Yes |
| Gestation rate | Yes |
| Piglet viability rate at separation | Yes |
| e. Mortality | Yes |
| Behaviour: | |
| a. Agitation level | Yes |
| b. Agression level | Yes |
| Disease presence | |
| a. Types | PRRS (pig) |
| b. Incidences | Yes |
| c. c. Time | Yes |

TABLE 5
Numbers of piglets farm 1
Farm 1

| Total number of animals | Tested animals | Control |
|---|---|---|
| 1784 | 839 | 945 |

TABLE 6
Number of growing up animals farm 2
Farm 2

| Total number of animals | Tested animals | Control |
|---|---|---|
| 1066 | 485 | 581 |

### Results

TABLE 7
Piglets, weight gain in nursery

| | EVPP tested with Formula of | | | |
|---|---|---|---|---|
| | wintergreen, cinnamon, and | | | |
| | licorice | | Control Group | |
| Entry weight | 5.55 | kg | 5.56 | kg |
| Exit weight | 29.25 | kg | 24.21 | kg |
| Number of days | 54 | | 54 | |
| Gain/day | 438 | g | 345 | g |

| | | |
|---|---|---|
| % mortality | 4.98% | 9.62% |

TABLE 8
Weight gain during growth

| | EVPP tested with Formula of | | | |
|---|---|---|---|---|
| | wintergreen, cinnamon, and | | | |
| | licorice | | Control Group | |
| Entry weight | 21.67 | kg | 21.88 | kg |
| Exit weight | 108.90 | kg | 107.12 | kg |
| Number of days | 92 | | 94 | |
| Index | 110.04 | | 109.80 | |
| % mortality | 4.59% | | 11.90% | |
| Gain/day | 948 | g | 907 | g |

### Conclusion and Discussion

The results above confirm the pertinence of controlling the inflammation in the pig with dietary supplement composition of the present invention. It appears that the pigs are in chronic inflammation, which could cause several disease resistance problems. The results show that the group receiving the dietary supplement composition of the present invention has a lower inflammatory protein level than the control group. Also, the group receiving the dietary supplement composition of the present invention shows an increased weight gain effect (See Tables 7 and 8).

### Example 2

### Effects of Polyphenols from Winter Green Oil, Pepper Mint, and Cinnamon as Feed Additive for Grain Veal Stress Control

### Materials & Methods

Ingredients and formulations: the same as in Example 1.

EVP (B)=bovine plant anti-inflammatory supplement (B)

### Experimental Protocol

As in Example 1.

TABLE 9
Concentrations

| Animals | Quantity |
|---|---|
| Milk veals | 2 g/day |
| Grain veals | 1 g/day |

The measurements parameters are as in Example, excepted for the blood tests.

### Results

TABLE 10
Number of animals per group
Farm

| Total number of animals | Tested animals | Control |
|---|---|---|
| 1357 | 935 | 422 |

The farms on which the experimentation was performed had a rotation of 2 for the two groups tested. Each test was repeated 12 times in the year.

TABLE 11
Weight gain (2 gr/day/veal)

| | |
|---|---|
| EVPB | Control Group |

| Entry weight | 130.66 | kg | 131.18 | kg |
|---|---|---|---|---|
| Exit weight | 349.7 | kg | 344.9 | kg |
| Number of days | 145 | | 145 | |
| Conversion | 3.2 | | 3.62 | |
| % mortality | 5.52% | | 10.55% | |
| Gain/day | 1.51 | kg | 1.47 | kg |

### Conclusion

The zootechnical results show that the calves responded similarly to the pigs of example 1, with a different dietary supplement composition of the present invention. Food conversion appears improved. Thus, an animal having received the dietary supplement composition of the present invention appears to have a digestive system in optimum mode of digestion.

### Example 3

### Impacts of the Incorporation of Dietary Supplement in the Dry Feed Ration on Primary and Secondary Performance During Lactation in a Holstein Dairy Herd

The polyphenols constitute a family of organic molecules widely present in the vegetal kingdom. They are characterized by the presence of at least two associated phenolic groups associated with more or less complex structures, typically of high molecular weight. These compounds are the products of the secondary metabolism of plants. Polyphenols are becoming increasingly important, especially thanks to their alleged beneficial effects on human health. Indeed, their role as natural antioxidants arouses growing interest in the prevention and treatment of cancer, of inflammatory, cardiovascular and neurodegenerative diseases. Over the past sixty years, the use of antibiotics in animal production has been increasingly important, not only to address animal health problems, but also to boost animal growth and production. This overuse of antibiotics has caused the emergence of antibiotic resistant bacterial strains, a situation of increasing concern, not only in animal health but also in human health. Faced with this situation, all health authorities (human and animal) have agreed to set up a very strict supervision of the use of antibiotics which should spell the end of their massive use. This evolution of the animal health approach will allow the diffusion of alternative approaches rather oriented towards the preventive than the curative with the use of molecules and/or active ingredients components other than antibiotics. Although several of these approaches were known, generally at laboratory level, they have never been widely disseminated because of the pressure from large pharmaceutical groups producing antibiotics. The use of polyphenols is one of these alternative approaches but the effects of these compounds from secondary metabolism of plants on animal health are not well documented, particularly in a preventive approach.

In a Holstein dairy herd, the incorporation of dietary supplement composition of the present invention into the dry period feed ration (60 days before calving=drying+preparation) has a significant positive impact on primary and secondary performance during the first 5 months of lactation. Indeed, in the experimental test group, compared to the control group, there were statistically significant improvements in primary performance (milk=+14%, fat=+18%, protein=+14%) but especially in health status (particularly in the immediate postpartum period) and fertility (+25%). These results support the hypothesis of the control of the anti-inflammatory phenomenon by a dietary supplement composition of the present invention (reduction of the infectious state→improvement of the state of health→improvement of milk, fat, protein; reduction of the inflammatory state of the uterine mucosa→improvement of fertility). The incorporation of dietary supplement composition of the present invention into the dry period feed ration of a Holstein dairy herd provides significant control of the inflammatory phenomenon and significantly reduces the physiological trauma of calving. The cow can thus start her lactation in a very good state of health and express her full dairy and reproductive potential. On the economic side, the preliminary economic cost-benefit analysis shows that the incorporation of dietary supplement composition of the present invention into the dry period feed ration of a Holstein dairy herd results in a net gain per cow and per lactation of $943.

### Materials & Methods

Two iso-parity experimental groups of twenty-six (26) Holstein-breed dairy cows (conventional management and robotic milking) were followed from the 60th day before calving until the end of the 5th month of lactation. The basic food ration of the “trial” group was supplemented with dietary supplement composition of the present invention at a rate of 50 g/day/cow from the start of dry-off (60th day before calving) until calving. The ingredients of the formulation of the present invention are the same as in Example 1. The food ration of the “control” group was the usual unsupplemented basic ration. The two groups of cows received the same treatment during the dry period: intramammary antibiotic treatment and obturators of the sphincters.

The environmental parameters were identical for the two experimental groups (dry period and lactation). Lactation food rations were the same for both experimental groups. The parameters monitored (per fortnight) were as follows:
Dairy performance: daily individual average in kg milk/day, kg fat/day, kg protein/day for month 1 to month 5 of lactation (interval: ½ month).
Individual state of health by monitoring the conductivity of milk in mS/cm—arithmetic mean of the values for each quarter—from month 1 to month 5 of lactation (interval: ½ month).
Fertility (individual number of inseminations to obtain the next gestation).

Statistical processing of the data made it possible to produce:
Dairy performances (milk, fat, proteins) of each experimental group: arithmetic means fortnightly weighted and general weighted arithmetic averages with 5-month standard deviations.
State of health of each experimental group: weighted arithmetic averages per fortnight and general geometric means with standard deviations over 5 months.
Fertility of each experimental group: geometric means and standard deviations.

Statistical analysis of the results was carried out using a comparative analysis of the values [avg±standard deviation] and a one-way analysis of variance (statistically significant differences or not in between groups) with a risk threshold α=0.10.

### Results—Primary and Secondary Performance

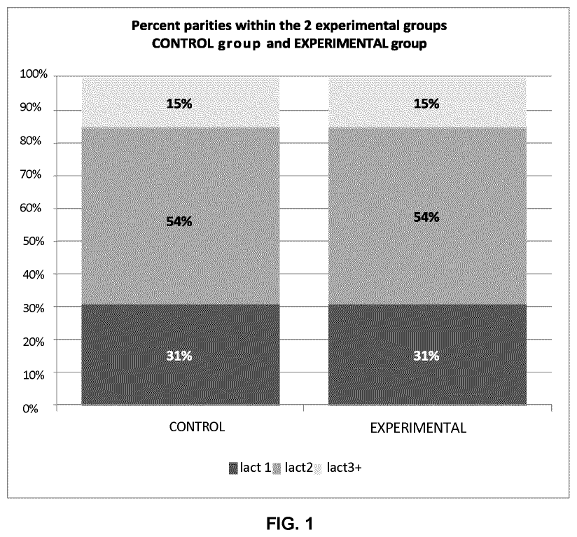

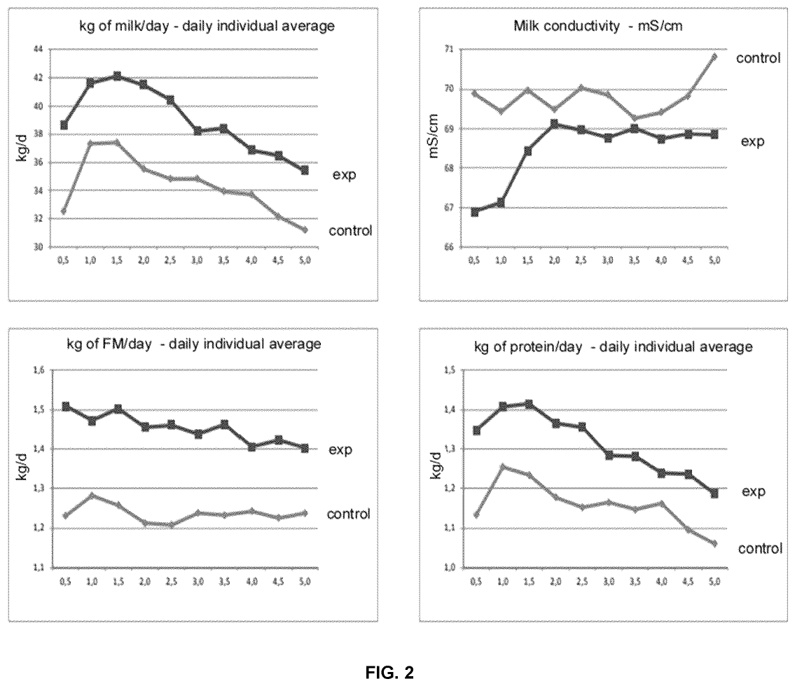

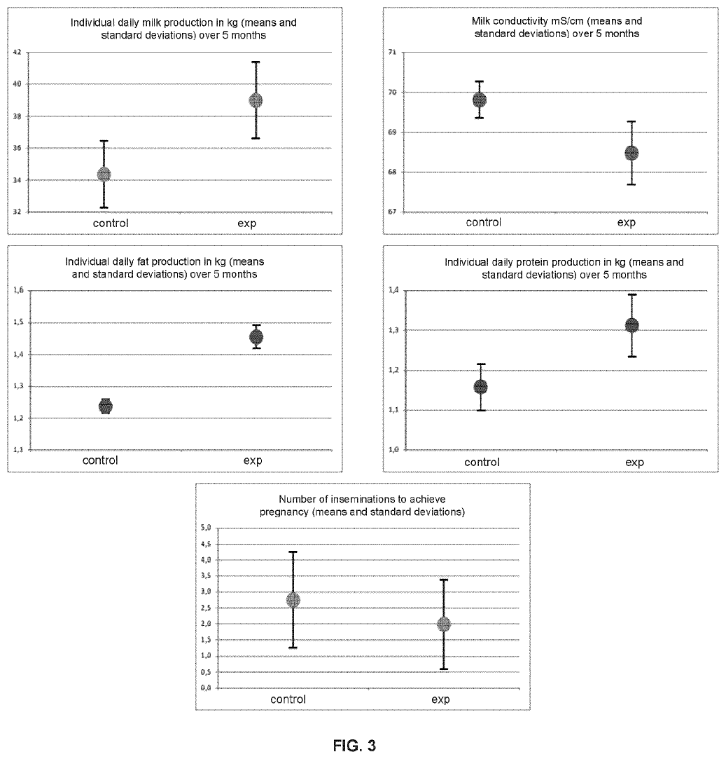

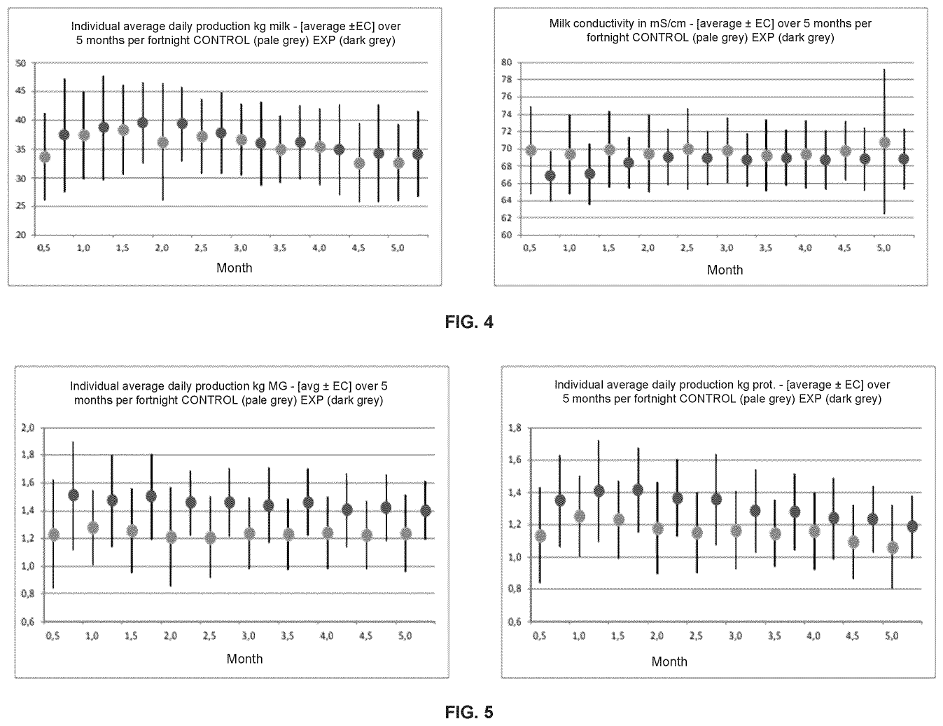

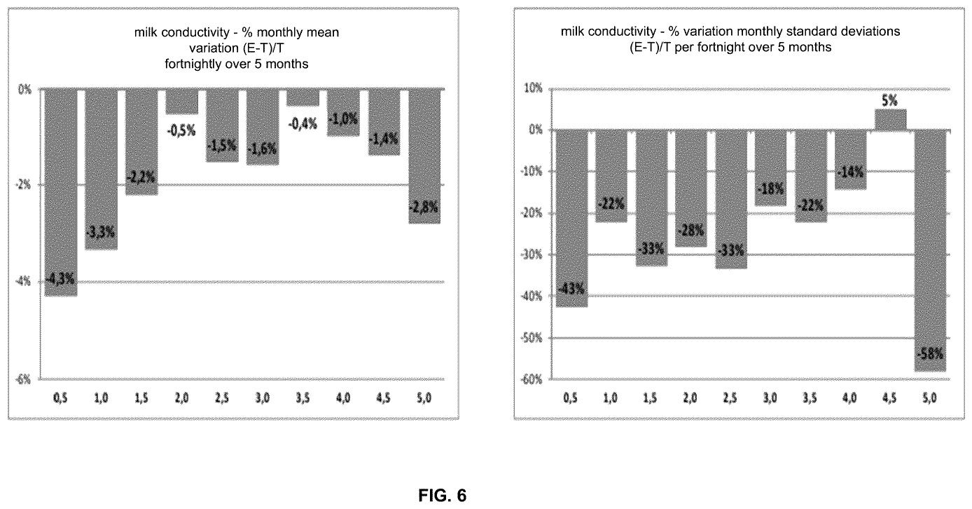

Table 12 shows the results obtained: milk, fat and proteins, milk conductivity and number of inseminations to obtain the next gestation. FIG. 1 shows the distribution of parities within the experimental groups. FIG. 2 illustrates the milk production (top left), fat (bottom left), protein (bottom left) and milk conductivity (top right) during the first 5 months of lactation. FIG. 3 illustrates the daily individual milk production performance over 5 months in kilograms and standard deviations (top left) and the fat matter content in kilograms and standard deviations (middle left), the conductivity (mS/cm) over 5 months (top right), the daily individual protein production performance over 5 months in kilograms and standard deviations (middle right) and fertility and standard deviations over 5 months (bottom). FIG. 4 illustrates Milk Production (left) and Milk Conductivity (right)-5-Month Monthly Averages and Standard Deviations. FIG. 5 illustrates Fat Production (left) and Protein Production (right)—5-Month Monthly Averages and Standard Deviations. FIG. 6 illustrates graphs of percentage of change in monthly mean milk conductivity and monthly standard deviations conductivity based on 5 months period per fortnight for cows fed with a composition of the present invention until calving (right) or not (left).

TABLE 12
Primary and secondary performance from month 1 to month 5 of lactation

| | Control Experimental group without PPH | | | | | | | | | | | |
|---|---|---|---|---|---|---|---|---|---|---|---|---|
| Month | 0.5 | 1.0 | 1.5 | 2.0 | 2.5 | 3.0 | 3.5 | 4.0 | 4.5 | 5.0 | | |
| kg/day | 32.55 | 37.33 | 37.40 | 35.55 | 34.84 | 34.85 | 33.96 | 33.73 | 32.16 | 31.22 | | |
| kg FM/d | 1.23 | 1.28 | 1.26 | 1.21 | 1.21 | 1.24 | 1.23 | 1.24 | 1.23 | 1.24 | | |
| kg prot/d | 1.13 | 1.26 | 1.23 | 1.18 | 1.15 | 1.17 | 1.15 | 1.16 | 1.10 | 1.06 | | |
| conduct. | 70 | 69 | 70 | 69 | 70 | 70 | 69 | 69 | 70 | 71 | | |
| | Experimental group with PPH | | | | | | | | | | | |
| Month | 0.5 | 1.0 | 1.5 | 2.0 | 2.5 | 3.0 | 3.5 | 4.0 | 4.5 | 5.0 | | |
| kg/d | 38.68 | 41.63 | 42.13 | 41.52 | 40.47 | 38.26 | 38.43 | 36.88 | 36.47 | 35.42 | | |
| kg MF/d | 1.51 | 1.47 | 1.50 | 1.46 | 1.46 | 1.44 | 1.46 | 1.41 | 1.42 | 1.40 | | |
| kg prot/d | 1.35 | 1.41 | 1.41 | 1.37 | 1.36 | 1.29 | 1.28 | 1.24 | 1.24 | 1.19 | | |
| conduct. | 67 | 67 | 68 | 69 | 69 | 69 | 69 | 69 | 69 | 69 | | |
| | kg milk/day | | | | | | | | | | | |
| Month | 0.5 | 1.0 | 1.5 | 2.0 | 2.5 | 3.0 | 3.5 | 4.0 | 4.5 | 5.0 | avg | sd |
| Control | 32.55 | 37.33 | 37.40 | 35.55 | 34.84 | 34.85 | 33.96 | 33.73 | 32.16 | 31.22 | 34.36 | 2.07 |
| Exp | 38.68 | 41.63 | 42.13 | 41.52 | 40.47 | 38.26 | 38.43 | 36.88 | 36.47 | 35.42 | 38.99 | 2.36 |

Comparison of Exp in relations to Control 13.5%

| | kg FM/day | | | | | | | | | | | |
|---|---|---|---|---|---|---|---|---|---|---|---|---|
| Month | 0.5 | 1.0 | 1.5 | 2.0 | 2.5 | 3.0 | 3.5 | 4.0 | 4.5 | 5.0 | avg | sd |
| Control | 1.23 | 1.28 | 1.26 | 1.21 | 1.21 | 1.24 | 1.23 | 1.24 | 1.23 | 1.24 | 1.24 | 0.02 |
| Exp | 1.51 | 1.47 | 1.50 | 1.46 | 1.46 | 1.44 | 1.46 | 1.41 | 1.42 | 1.40 | 1.45 | 0.04 |

Comparison of Exp in relations to Control 17.5%

| | % FM | | | | | | | | | | | |
|---|---|---|---|---|---|---|---|---|---|---|---|---|
| Month | 0.5 | 1.0 | 1.5 | 2.0 | 2.5 | 3.0 | 3.5 | 4.0 | 4.5 | 5.0 | | |
| Control | 3.78% | 3.44% | 3.36% | 3.41% | 3.47% | 3.55% | 3.63% | 3.69% | 3.81% | 3.97% | | |
| Exp | 3.90% | 3.54% | 3.57% | 3.51% | 3.61% | 3.76% | 3.81% | 3.81% | 3.91% | 3.96% | | |
| | kg Proteins/day | | | | | | | | | | | |
| Month | 0.5 | 1.0 | 1.5 | 2.0 | 2.5 | 3.0 | 3.5 | 4.0 | 4.5 | 5.0 | avg | sd |
| Control | 1.13 | 1.26 | 1.23 | 1.18 | 1.15 | 1.17 | 1.15 | 1.16 | 1.10 | 1.06 | 1.16 | 0.06 |
| Exp | 1.35 | 1.41 | 1.41 | 1.37 | 1.36 | 1.29 | 1.28 | 1.24 | 1.24 | 1.19 | 1.31 | 0.08 |

Comparison of Exp in relations to Control 13.3%

| | % Proteins | | | | | | | | | | | |
|---|---|---|---|---|---|---|---|---|---|---|---|---|
| Month | 0.5 | 1.0 | 1.5 | 2.0 | 2.5 | 3.0 | 3.5 | 4.0 | 4.5 | 5.0 | | |
| Control | 3.48% | 3.36% | 3.30% | 3.32% | 3.31% | 3.34% | 3.38% | 3.44% | 3.41% | 3.40% | | |
| Exp | 3.48% | 3.38% | 3.36% | 3.29% | 3.35% | 3.36% | 3.33% | 3.36% | 3.39% | 3.35% | | |
| | conductivity of milk mS/cm | | | | | | | | | | | |
| Month | 0.5 | 1.0 | 1.5 | 2.0 | 2.5 | 3.0 | 3.5 | 4.0 | 4.5 | 5.0 | avg | sd |
| Control | 70 | 69 | 70 | 69 | 70 | 70 | 69 | 69 | 70 | 71 | 69.80 | 0.45 |
| Exp | 67 | 67 | 68 | 69 | 69 | 69 | 69 | 69 | 69 | 69 | 68.48 | 0.80 |

Comparison of Exp in relations to Control −1.9%

| | Number of inseminations for gestation | | | |
|---|---|---|---|---|
| | max | min | avg | sd |
| Control | 6.00 | 1.00 | 2.74 | 1.49 |
| Exp | 5.00 | 1.00 | 1.99 | 1.38 |

Comparison of Exp in relations to tem: −27.6%
PPH = polyphenol treatment;
FM = fat matter;
AVG = average;
sd = standard deviation Control = control group; Exp = Experimental Group ;

As described in Example 4, the percentages of variation are calculated as follows:
% change in monthly mean conductivity=[(avg WPPH−avg W/O PPH)/(avg W/O PPH)]
% variation monthly standard deviations conductivity=[(sd WPPH−sd W/O PPH)/(sd W/O PPH)]
WPPH=with dietary supplement composition of the present invention; W/O PPH=dietary supplement composition of the present invention.

### Variance Analysis Results

The results of the 1-way analysis of variance (a composition of the present invention) with a risk threshold α=0.10 are presented in the table below.

TABLE 13
Results of 1-way analysis of variance - Primary and secondary performance

| P[Fcritical > | | | % |
|---|---|---|---|
| Fobserved] | Decision (α = 0.10) | | (E − T)/T |
| 2.31445E−11 | Decision Treatment effect with | significant positive impact | +14.08% |
| | significant difference | on milk | |
| 7.94476E−17 | Decision Treatment effect with | significant positive impact | +17.75% |
| | significant difference | on MF | |
| 8.67441E−12 | Decision Treatment effect with | significant positive impact | +13.87% |
| | significant difference | on protein | |
| 6.02049E−06 | Decision Treatment effect with | significant positive impact | −2.40% |
| | significant difference | on conductivity of milk | |
| 0.000812991 | Decision Treatment effect with | significant positive impact | −3.92% |
| | significant difference | on conductivity month 1 | |
| 0.048731379 | Decision Treatment effect with | significant positive impact | 25.75% |
| | significant difference | on fertility | |

The results show statistically significant positive effect. Fertility is quantified in number of insemination(s) to obtain a gestation.

### Discussion

Analysis of the results suggests that the incorporation of dietary supplement composition of the present invention in the food ration during the dry period (D-60→calving), during the first 5 months of lactation results in a statistically significant improvement in the production of milk (+14%), fat (+18%) and proteins (+14%), a statistically significant improvement in the conductivity of milk of −2.4% with an even more marked statistically significant improvement of −3.9% during the first month of lactation, that is to say the immediate postpartum, which is a period of high health risk, and a statistically significant improvement in fertility of −26% (counted in number inseminations to obtain gestation).

Regarding the state of health (measured by the conductivity of the milk-indicator of the infectious state), the incorporation of a dietary supplement composition of the present invention in the food ration of the dry period results not only in a significant improvement in the average state of health of the trial group but also by a much less marked dispersion around the average values during the first 5 months of lactation. This reflects an improvement in the health of the entire group with dietary supplement composition of the present invention.

### Preliminary Economic Analysis

The economic analysis aims to determine whether the incorporation of dietary supplement composition of the present invention in the diet of the dry period generates a benefit significantly greater than the cost of the dietary supplement composition of the present invention itself. Calculations presented below correspond to a dairy farm with a quota of 158 kg fat/day.

TABLE 14
Cost/benefit analysis-Primary performance

| Paye de | | kg FM/day/cow | | | | | average |
|---|---|---|---|---|---|---|---|
| lait | Month | 1 | 2 | 3 | 4 | 5 | lactation |
| FM | W/O PPH | 1.28 | 1.21 | 1.24 | 1.24 | 1.24 | 1.24 |
| | WPPH | 1.47 | 1.46 | 1.44 | 1.41 | 1.40 | 1.41 |
| | $/kg FM | 10.6380 $ | average 2018 price | | | | |
| $ FM | W/O PPH | 408.50 $ | 386.16 $ | 395.73 $ | 395.73 $ | 395.73 $ | 3 957 $ |
| | WPPH | 469.13 $ | 465.94 $ | 459.56 $ | 449.99 $ | 446.79 $ | 4 484 $ |
| | Δ $FM/lactation | | | | | | 527 $ |
| | | kg of proteins/day/cow | | | | | average |
| | Month | 1 | 2 | 3 | 4 | 5 | lactation |
| Proteins | W/O PPH | 1.26 | 1.18 | 1.17 | 1.16 | 1.06 | 1.11 |
| | WPPH | 1.41 | 1.37 | 1.29 | 1.24 | 1.19 | 1.22 |
| | $/kg prot | 7.1011 $ | average 2018 price | | | | |
| $ proteins | W/O PPH | 268.42 $ | 251.38 $ | 249.25 $ | 247.12 $ | 225.82 $ | 2 365 $ |
| | WPPH | 300.38 $ | 291.86 $ | 274.81 $ | 264.16 $ | 253.51 $ | 2 588 $ |
| | Δ $prot/lactation | | | | | | 224 $ |

| Milk pay/cow | | |
|---|---|---|
| Food ration | Average lactation | Month average |
| W/O PPH | 6 322 $ | 632 $ |
| WPPH | 7 072 $ | 707 $ |
| Δ $ (WPPH-W/O PPH) | 750 $ | 75 $ |
| Δ $ en % | 12% | |

note:
lactose + AS and SNG not measured. Considered equal for both experimental groups.

### Annual Estimate for all Suckling Livestock-Milk Pay Income

quota=158 kg/FM/day
average number of suckler cow=150 and 10 month of lactation per cow.
Number of month of lactating cow/year=1896

TABLE 15
Improvement of paid milk revenue generated by livestock

| paid milk revenue generated | |
|---|---|
| by livestock - projection | total per year |
| dry ration p without PPH | 1 198 650 $ |
| dry ration p with PPH | 1 340 900 $ |
| Δ $ (WPPH − W/O PPH) | 142 250 $ |
| Δ $ in % | 12% |

TABLE 16
Cost/benefit analysis - Fertility and synthesis

| | average number of | | | | |
|---|---|---|---|---|---|
| | inseminations for pregnancy | | | | |
| | Individual | | | | |
| | performance | | suckling livestock | | insemination |
| fertility performance | W/O PPH | WPPH | W/O PPH | WPPH | unit cost |
| No of insemination | 3.16 | 2.35 | 499 | 371 | 75.00 $ |
| per pregnancy | | | | | |
| Δ insemination | | −26% | | | |
| in % | | | | | |

| | Suckling livestock | | Costs | | Δ $ |
|---|---|---|---|---|---|
| fertility performance | SPPH | APPH | W/O PPH | WPPH | (APPH − SPPH) |
| No. of total | 499 | 371 | 37 446 $ | 27 802 $ | (9 644) $ |
| insemination | | | | | |

The above results are significantly positive. Note that the analysis did not take into account the positive economic impacts of the reduction in the number of subclinical and clinical mastitis as well as reducing the interval between calving.

### Conclusion

The incorporation of dietary supplement composition of the present invention in the dry period feed ration of a Holstein dairy herd allows to obtain a significant control of the inflammatory phenomenon and to considerably reduce the physiological trauma of calving. The cow can thus start her lactation in a very good state of health and express its full dairy and reproductive potential. Economically, the economic analysis preliminary cost vs benefit demonstrates that the incorporation of a composition of the present invention in the diet of the dry period of a Holstein dairy herd results in a net gain per cow and per lactation of $943.

### Example 4

### Clinical Trial on Old Dog Suffering from Osteoarthritis Receiving Scientifically Formulate Natural Polyphenols Therapeutic Treat

One of the most prevalent musculoskeletal disorders in old dog is osteoarthritis. Osteoarthritis is a chronic inflammation disease mainly related to aging and genetic weakness associated to some large breed. The locomotive system of the animal is affected a way that inflammation become chronic and the dog express pain and reduce physical activities. So far, the majority of conventional treatment (meloxicam, cartrophen, derecoxib) are not permanent and pleasant to administer and received by the animal.

Since major progress are accomplished in improving nutrition to extend quality life there is more shifted from focusing exclusively on alleviating nutrient deficiencies to also stressing chronic disease prevention. Therefore, a clinical trial was design to verify if old, obese and big breed of dog can benefit from administration of a dietary supplement composition of the present invention. A special treat was design with high concentration of those unique formulation of the present invention to see if sick dogs will respond and if this will improve quality of life by reducing inflammation due to osteoarthritis.

### Introduction: Inflammation

Inflammation is a vital part of the immune system's response to injury and infection. It is the body's way signaling the immune system to heal and repair damaged tissues, as well as defend itself against foreign invaders, such as virus and bacteria. Without inflammation as a physical response, wound would fester, and infections could become deadly. However, if the inflammatory process goes too long or if it occurs at places not needed, it become problematic. Chronic inflammation has been linked to certain autoimmune disease such as rheumatoid arthritis. But healthy diet and lifestyle can help keep inflammation under control. There is two type of inflammation, acute and chronic inflammation. Acute inflammation occurs on direct stimulus and trigger pain and fever as part of the healing process. However chronic inflammation is considered a disturb permanent reaction that trigger long term disease such as arthritis.

### Anti-Inflammatory Diet or Dietary Supplement and Anti-Inflammatory Drug

Anti-inflammatory diet and supplement have become popular in recent year, however it was observed that very few diet or supplement look very efficient in a short term basis. Professional such as veterinarians seek short term solutions to help control the pain of the animal. In both cases, the solution doesn't solve the problem, but only mitigates the disease. The standard treatment should always be kept in consideration by the risk of adverse effects caused by long-term toxicity of drugs such as nonsteroidal anti-inflammatory drugs.

The focus of this example is to phenotypically evaluate if a dietary supplement composition of the present invention can help reduce inflammation and provide a positive response by reducing pain and improve quality of life.

This trial is based on the premise of optimum welfare by reducing stress factors on companion animal such as blood sampling by using numerous blood sampling already done on livestock. The physiology of inflammation is considered to be very similar in dog and other species. Therefore, the previous study in livestock demonstrated the use of this precise dietary supplement composition in pain control management and stress reduction in acute stress challenge.

### Materials & Methods

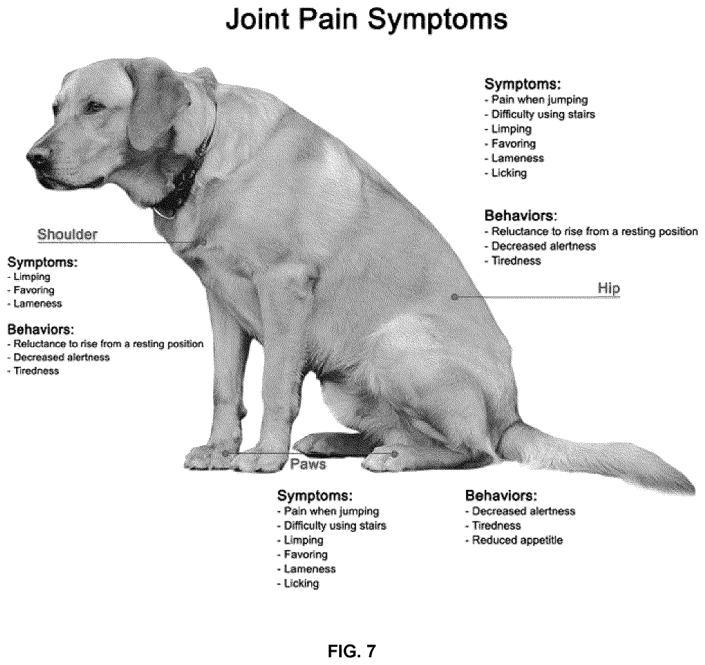

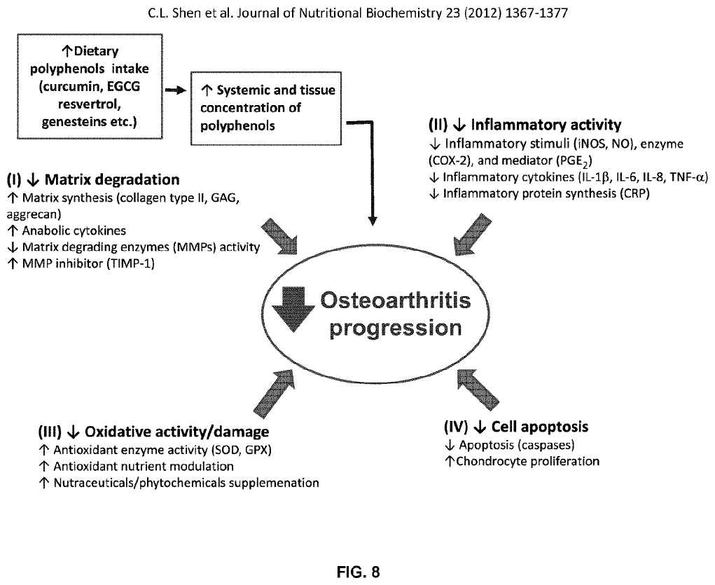

Animals: dogs: 8 adult dogs (>5 years old), that weigh >20 kg, male or female and with various medical history are used in our trial, also a minimum of 8 dogs that show signs of osteoarthritis (i.e. lameness, see FIGS. 7 and 8) are used. They must not receive any drug or treatment prior and/or during the trial. Pregnant females or lactating females will be excluded for the trial.

### Composition of the Present Invention

The approach of the clinical study was to provide a specific blend of the dietary supplement composition of the present invention. The composition comprises about 2.5% w/w of wintergreen oil, about 0.133% w/w of peppermint essential oil, about 0.005% w/w carvacrol, about 1.087% w/w cinnamaldehyde, about 0.012% w/w ginger essential oil, about 0.012% w/w anise essential oil, and about 0.048% w/w liquorice (comprising 33% w/w glycyrrhizin). To prepare the treat, an actual high-quality treat formula was used and supplemented with the dietary supplement composition of the present invention. The treat contains real meat, potato meal and other type of meal that we mix with the blend and expose to high pressure and high temperature in a standard Petfood extruder. Three size and shape of treat were produced to see animal preference. The small star shape was the preferred of large dog.

### Criteria for Canine Osteoarthritis

The efficacy of the special treat will be evaluated based on: 1) Evaluation of phenotypic changes 2) Locomotive response of the companion animal. Animals will be graded using Lameness scoring system (Table 18). Lameness is defined as a deviation from the normal gait or locomotion of an animal and is most commonly due to an abnormality of the musculoskeletal system. 3) the percentage of dogs showing improvement. 4) interval between the beginning of the trial and the improvement and 5) the amplitude of improvement.

TABLE 18
Clinical score

| PARAMETER | SCORE | DESCRIPTION OF SCORE |
|---|---|---|
| Lameness | 1 | Walks normally |
| | 2 | Slightly lame when walking |
| | 3 | Moderately lame when walking |
| | 4 | Severely lame when walking |
| | 5 | Reluctant to rise and will not walk more than five paces |
| Joint mobility | 1 | Full range motion |
| | 2 | Mild limitation (10-20%) in range of motion; no crepitus |
| | 3 | Mild limitation (10-20%) in range of motion ; crepitus |
| | 4 | Moderate limitation (20-50%) in range of motion; +/−crepitus |
| | 5 | Severe limitation (+50%) in range of motion ; +/−crepitus |
| Pain on palpation | 1 | None |
| | 2 | Mild signs; dog turns pulls limb away |
| | 3 | Moderate signs; dog pulls limb away |
| | 4 | Severe signs; dog vocalizes or becomes aggressive |
| | 5 | Dog will not allow palpation |
| Weight bearing | 1 | Equal on all limbs standing and walking |
| | 2 | Normal standing; favors affected limb when walking |
| | 3 | Partial weight-bearing standing and walking |
| | 4 | Partial weight-bearing standing; non-weight-bearing walking |
| | 5 | Non-weight-bearing standing and walking |
| Overall score | 1 | Not affected |
| of clinical | 2 | Mildly affected |
| condition | 3 | Moderately affected |
| | 4 | Severely affected |
| | 5 | Very severely affected |

### Clinical Observations and Measurements

These elements shall be registered for each dog: name/breed/age/color/sex/weight/. Each dog is selected based on pet owner that saw significant change in the animal behavior. Observation of initial improvements, physical examinations will be done by owner. Each dog will be assessed for its general health and presence of problem of any origin. Each dog that shows signs of lameness will be taken in picture. Any relevant comments will be added to the file. Physical examination includes, without being exclusive: —General appearance—Musculoskeletal examination. Any medication used and the reason for its use should be documented during the trial period and communicated as soon as possible. Any dog can be removed or replaced during the first 2 weeks of the test (no more than 25%) due to a deficient consumption, the reasons for which must be recorded.

### Dose Response

The determination of the dose-response of the therapeutic dog treat were establish with 2 criterion:
1) Welfare issue to minimize stress on old dog by keep them in their own environment and to avoid blood sampling on a phase one.
2) Previous experimental studies done on pigs, cows and broiler birds. The dose was established in relation to body weight and metabolic rate of 5 years old, big breed dog versus the evaluation of the different dose response of livestock.

TABLE 19
mg/dietary supplement composition activity based
on multi species analogy with a liquid supplement

| LIQUID | | | | |
|---|---|---|---|---|
| SUPPLEMENT | Pig | Poultry | Cow | Dog |
| Nb animals | 33,600 | 880,000 | 2,160 | 2 |
| Nb years | 8 | 8 | 4 | 6 |
| Nb of replicate | 150 | 80 | 25 | 1 |
| Metabolic rate | 1 | 1.2 | .8 | .6 |
| evaluation | | | | |
| multiplier | | | | |
| Efficiency % | 100% | 120% | 80% | 60% |
| Mg/kg body | 900 mg/kg | 750 mg/kg | 1125 mg/kg | 1500 mg/kg |
| weight per day | | | | |

TABLE 20
mg/dietary supplement composition
activity on multi species with dry supplement

| DRY SUPPLEMENT | Pig | Poultry | Cow | Dog |
|---|---|---|---|---|
| Nb animals | 122,000 | 8 millions+ | 12,000+ | 8 |
| Nb years | 8 | 8 | 4 | 1 |
| Nb replicate | 6 | 16 | 20 | 1 |
| Metabolic rate | 1 | 1.2 | 8 | 6 |
| evaluation multiplier | | | | |
| Efficiency % | 100% | 120% | 80% | 60% |
| Mg/kg body weight | 1500 | 1200 | 1875 | 2500 |
| per day | mg/kg | mg/kg | mg/kg | mg/kg |

### Quantity of Supplement

Each dog will be received 125 grams treat per day by 40 kg body weight for the duration of the test.

### Duration

The determination of the duration of the trial was based on previous livestock data response, slower metabolic rate of companion animal vs livestock and two weeks delay response was sufficient time base on the two previous criteria.

TABLE 21
Previous response speed in livestock:
Type of response: body temperature

| | Speed | | | | | | |
|---|---|---|---|---|---|---|---|
| | in hr | Pig | | Poultry | | Cow | |
| Liquid | Rapid | 20 | minutes | 20 | minutes | 1 | hr |
| supplement | Mean | 1 | hr | 30 | min | 1.5 | hr |
| | Slowest | 2 | hr | 1 | hr | 4 | hr |
| Dry feed | Rapid | 1 | day | 1 | day | 3 | days |
| | Mean | 2 | days | 1 | day | 5 | days |
| | Slowest | 5 | days | 2 | day | 21 | days |

Note: Circadian cycle was considered, and control didn't express any notable change.

### Duration of the Test

The trial was run for a minimum of 2 weeks and began as soon as the dogs were fed the supplement. According to the results of the first 2 weeks, the trial may be continued or not.

### Food and Feeding Parameters

Food must stay the same during the trial. Fresh water must be available at all time for every dog. Dog may be fed ad libidum or according to their nutritional needs. Any termination or modification of the food protocol must be described and reported as soon as possible.

### Handling and Environment

Enclosures and bowls shall be cleaned daily (or as needed). Dogs should be fed twice a day and clean, fresh water shall be available at all time. Dogs shall have a daily period of physical activity

### Statistical Analysis

The clinical sign scores were calculated as mean±SD. Nonparametric 2-sample Mann-whitney procedure was used to test for the differences before and after treatment. Relative data were analyzed using the SAS version 8.0 software package; P≤0.01 was considered to be significant. The study design had several limitations. Because this was a clinical study the animals could not be controlled by using the same breed, sex, and age. The other limitation is the scores evaluation where it may vary in interpretation of each animal.

### Material

Camera, Thermometer, watch, stethoscope, Razor, Isopropylique alcohol, Gauzes, Needles+syringes+5 ml blood tubes (purple and red), Cooler, Centrifuge, Physical examination sheets, Pen.

Results

TABLE 23
Response speed in hour and days

| | 1 | 2 | 3 | 4 | 5 | 6 | 7 |
|---|---|---|---|---|---|---|---|
| Dog 1 | | | | | X | | |
| Dog 2 | | X | | | | | |
| Dog 3 | | X | | | | | |
| Dog 4 | | | X | | | | |
| Dog 5 | | X | | | | | |
| Dog 6 | | X | | | | | |
| Dog 7 | | | X | | | | |
| Dog 8 | | | X | | | | |

TABLE 24
Response speed in hour and days

| | 1 | 2 | 3 | 4 | 5 | 6 | 7 |
|---|---|---|---|---|---|---|---|
| Dog | | 4 | 3 | | 1 | | |

TABLE 25
Response to treatment

| | NO | YES |
|---|---|---|
| Dog 1 | | X |
| Dog 2 | | X |
| Dog 3 | | X |
| Dog 4 | | X |
| Dog 5 | | X |
| Dog 6 | | X |
| Dog 7 | | X |
| Dog 8 | | X |

TABLE 26
Response to treatment

| | NO | YES |
|---|---|---|
| Dogs | 0/8 | 8/8 |

TABLE 27
Satisfaction of treatment: 5 = very satisfied and 1 = not satisfied

| | 1-5 | REMARKS |
|---|---|---|
| Dog 1 | 5 | Before the treat, Maggie was not able to climb the sofa or the stairs. She |
| | | didn't want to go out for urinating. She was not walking, unless she was |
| | | forced to walk. The hips were full of inflammation. After 7 days of daily |
| | | therapeutic treat, Maggie is now climbing the sofa, asks to go out for her |
| | | need and show normal physical activities. My dad and I are very impressed |
| | | with the speed of healing. |
| Dog 2 | 5 | Before the treat, Princesse Sissi was very lazy to move and showed steady |
| | | pain by crying every time she needed to move. The hips and leg were |
| | | chronically inflamed and her activities were very limited. After the |
| | | treatment she stoped crying and she is doing normal activities without any |
| | | trouble or showing weakness. After 3 weeks of treatment, we had stopped |
| | | to use the treat and Princesse is still ok. |
| Dog 3 | 5 | Before the treat Sandy was having serious problem to climb the stairs. Her |
| | | hips were showing inflammation and she was having trouble to walk. After |
| | | the treat she almost walk normally and show active behavior. |
| Dog 4 | 5 | Before the treat, Mandy was declared by two veterinary clinics to be |
| | | euthanized. She was having serious problem of locomotion and she was |
| | | crying all the time. After the treatment with the treat, within 2 days we |
| | | started to see significant response from Mandy. After a week she was |
| | | climbing the stairs and running in the yard. Mandy lived 2 more full years. |
| Dog 5 | 5 | Before the treat, Boddy was not very active because she was having |
| | | problem walking. After the treatment with the treat, she now runs, walks |
| | | and plays with the kids again. Our dog response very rapidly to the treat. |
| | | We noticed when we stopped feeding the daily treat, the locomotive |
| | | problem started to show again. But it took several days before showing |
| | | walking problem. So now we use the treat every day. |
| Dog 6 | 5 | Before the treat, Samy was an old lazy male dog doing nothing all day long. |
| | | Samy was crying every time that he needed to move or go out. After a few |
| | | days of treatment with the treat, Samy was much more active and was |
| | | playing with us in the backyard. We have fed this daily treat for the last 2 |
| | | years of life of my Samy, Samy pass away at the age of 15 years, which is |
| | | old for a Labrador. |
| Dog 7 | 5 | Before the treat, Booggy was an old male and not a very active dog. Booggy |
| | | was walking with pain and was not anymore running. Booggy was also |
| | | having serious skin problem with chronic inflammation. After the use of |
| | | the treat, Booggy is almost running and walking normally. Booggy is just |
| | | slower. After 10 days of treatment the skin inflammation problem |
| | | disappeared. |
| Dog 8 | 5 | Before the treat, Theo was having problem to walk, run and was showing |
| | | steady inflammation on the hips. After 2 days our dog is running again and |
| | | walking. We were surprised about the speed of response and |
| | | improvement of our dog health. |

TABLE 28
Satisfaction of treatment

| | 5 | Commun observations |
|---|---|---|
| Dog | 8/8 | Speed of improvement |

TABLE 29
Comparison of clinical scores for the osteoarthritis before and after

| | PARAMETER | BEFORE | AFTER |
|---|---|---|---|
| Dog 1 | Lameness | 4.5 | 1.0 |
| | Joint mobility | 4.0 | 2.0 |
| | Pain on palpation | 5.0 | 1.0 |
| | Weight bearing | 4.0 | 1.0 |
| | Overall score | 4.0 | 1.5 |
| Dog 2 | Lameness | 3.5 | 1.5 |
| | Joint mobility | 4.0 | 1.0 |
| | Pain on palpation | 4.5 | 1.0 |
| | Weight bearing | 4.0 | 1.0 |
| | Overall score | 4.5 | 1.5 |
| Dog 3 | Lameness | 4.0 | 1.25 |
| | Joint mobility | 4.0 | 1.0 |
| | Pain on palpation | 4.0 | 1.0 |
| | Weight bearing | 4.0 | 1.0 |
| | Overall score | 4.0 | 1.5 |
| Dog 4 | Lameness | 5.0 | 1.5 |
| | Joint mobility | 5.0 | 1.0 |
| | Pain on palpation | 5.0 | 1.0 |
| | Weight bearing | 5.0 | 1.0 |
| | Overall score | 5.0 | 1.0 |
| Dog 5 | Lameness | 3.5 | 1.25 |
| | Joint mobility | 4.0 | 1.0 |
| | Pain on palpation | 4.0 | 1.0 |
| | Weight bearing | 4.0 | 1.0 |
| | Overall score | 3.5 | 1.25 |
| Dog 6 | Lameness | 4.5 | 1.25 |
| | Joint mobility | 5.0 | 1.0 |
| | Pain on palpation | 4.5 | 1.0 |
| | Weight bearing | 4.5 | 1.0 |
| | Overall score | 4.5 | 1.25 |
| Dog 7 | Lameness | 3.5 | 1.0 |
| | Joint mobility | 4.0 | 1.0 |
| | Pain on palpation | 3.5 | 1.0 |
| | Weight bearing | 3.5 | 1.0 |
| | Overall score | 3.75 | 1.0 |
| Dog 8 | Lameness | 4.0 | 1.0 |
| | Joint mobility | 4.0 | 1.0 |
| | Pain on palpation | 3.5 | 1.0 |
| | Weight bearing | 3.5 | 1.0 |
| | Overall score | 4.0 | 1.0 |

TABLE 30
Titre: Summary of comparison of clinical scores for osteoarthritis

| PARAMETER | BEFORE | AFTER |
|---|---|---|
| Lameness | 4 | 1.25 |
| Joint mobility | 4.25 | 1.25 |
| Pain on palpation | 4.25 | 1.25 |
| Weight bearing | 4 | 1.25 |
| Overall score | 4 | 1.25 |

Data are expressed as mean ± SD. A significant difference (p < 0.05) between the initial condition is displayed.

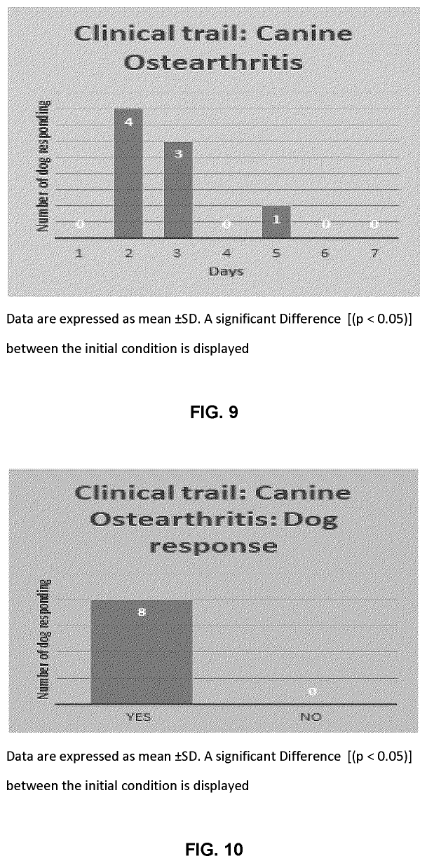

FIGS. 9 and 10 illustrate the speed of response and the number of animals that responded in the study.

### Discussion

The previous experimental trial performed with livestock was very helpful to reduce the number of companion animal to demonstrate the efficiency of the dietary supplement composition of the present invention to improve and treat osteoarthritis. All dog responded very well to the dietary supplement composition of the present invention. The results also show the speed of the response upon each animal and the quality life given back to each dog. The results obtained were even better than conventional treatment that was much more aggressive on animal. Furthermore, the speed of the response was unexpectedly much faster than all others type of nutrients such as glucosamine, chondroitin and MSM, which have little or no demonstrated efficacy.

### Example 5

### Species Specific Formulations

| | Bovine | Pork | Poultry | Horse |
|---|---|---|---|---|
| Ingredient | (% w/w) | (% w/w) | (% w/w) | (% w/w) |
| Wintergreen oil | 2.070 | 2.250 | 2.750 | 2.500 |
| (99%) | | | | |
| Peppermint | 0.110 | 0.012 | 0.146 | 0.133 |
| essential oil (99%) | | | | |
| Carvacrol 99% | 0.004 | 0.004 | 0.005 | 0.005 |
| Cinnamaldehyde | 0.900 | 0.978 | 1.196 | 1.087 |
| 98% | | | | |
| Ginger essential oil | 0.010 | 0.011 | 0.013 | 0.012 |
| 99% | | | | |
| Anise essential oil | 0.010 | 0.011 | 0.013 | 0.012 |
| 99% | | | | |
| Liquorice 98% (33% | 0.040 | 0.043 | 0.053 | 0.048 |
| glycyrrhizin) | | | | |
| chromium picolinate | 299 ppb | 299 ppb | 299 ppb | 299 ppb |

| | Canine | Feline | Sheep or Goat |
|---|---|---|---|
| Ingredient | (% w/w) | (% w/w) | (% w/w) |
| Wintergreen oil | 2.500 | 2.500 | 2.350 |
| (99%) | | | |
| Peppermint | 0.133 | 0.133 | 0.125 |
| essential oil (99%) | | | |
| Carvacrol 99% | 0.005 | 0.005 | 0.005 |
| Cinnamaldehyde | 1.087 | 1.087 | 1.022 |
| 98% | | | |
| Ginger essential oil | 0.012 | 0.012 | 0.011 |
| 99% | | | |
| Anise essential oil | 0.012 | 0.012 | 0.011 |
| 99% | | | |
| Liquorice 98% (33% | 0.048 | 0.048 | 0.045 |
| glycyrrhizin) | | | |
| chromium picolinate | 299 ppb | 299 ppb | 299 ppb |

While preferred embodiments have been described above and illustrated in the accompanying drawings, it will be evident to those skilled in the art that modifications may be made without departing from this disclosure. Such modifications are considered as possible variants comprised in the scope of the disclosure.

1. A livestock dietary supplement composition comprising:
a) from about 0.35% to about 4% w/w of a sialagogue comprising cinnamaldehyde;
b) from about 2% to about 10% w/w of an anti-inflammatory comprising a salicylate;
c) from about 0.1% to about 13% w/w of an expectorant comprising menthol;
d) from about 0.0000022% to about 0.04% w/w of an antimicrobial comprising carvacrol, anethole and a ginger essential oil; and
e) a sufficient amount of an acceptable carrier to make up to 100% w/w.

2. The livestock dietary supplement composition of claim 1, wherein said salicylate is methyl salicylate, magnesium salicylate, choline salicylate, bismuth subsalicylate, salsalate, salicin, salicylic acid, acetylsalicylic acid and combinations thereof.

3. The livestock dietary supplement composition of claim 1, wherein said salicylate is provided by a wintergreen essential oil.

4. The livestock dietary supplement composition of claim 1, wherein said expectorant further comprises menthone and eucalyptol.

5. The livestock dietary supplement composition of claim 1, wherein said expectorant comprises a peppermint essential oil.

6. The livestock dietary supplement composition of claim 1, wherein in said antimicrobial, said carvacrol is provided by a thyme essential oil, an oregano essential oil, a pepperwort essential oil, a wild bergamot essential oil, a Satureja essential oil, a marjoram essential oil, a Origanum Dictamnus essential oil, and combinations thereof.

7. The livestock dietary supplement composition of claim 1, wherein in said antimicrobial, said anethole is provided by an anise essential oil, a liquorice, a liquorice extract, and combinations thereof.

8. The livestock dietary supplement composition of claim 1, further comprising from about 2% to about 8% w/w of an astringent comprising an edible polyphenolic compound.

9. The livestock dietary supplement composition of claim 8, wherein said edible polyphenolic compound is a tannin.

10. The livestock dietary supplement composition of claim 1 further comprising from about 0,00001 to about 0,00005% w/w of a chromium.

11. The livestock dietary supplement composition of claim 10, wherein said chromium is chromium picolinate, an amino acid chelate of chromium, or combinations thereof.

12. The livestock dietary supplement composition of claim 1, further comprising a vitamin.

13. The livestock dietary supplement composition of claim 12, wherein said vitamin is vitamin C, vitamin D, vitamin E, folic acid, or combinations thereof.

14. The livestock dietary supplement composition of claim 1, wherein said anti-inflammatory further comprises a coumarin.

15. The livestock dietary supplement composition of claim 1, further comprising a thickening agent.

16. The livestock dietary supplement composition of claim 15, wherein said thickening agent is locust bean gum.

17. The livestock dietary supplement composition of claim 1, wherein said acceptable carrier comprises a clay, an aluminosilicate, and combinations thereof.

18. The livestock dietary supplement composition of claim 7, comprising:
a) from about 0.9% to about 1.2% w/w of cinnamaldehyde;
b) from about 2% to about 3% w/w of a wintergreen oil;
c) from about 0.1% to about 0.15% w/w of a peppermint oil;
d) from about 0.06% to about 0.085% w/w of an antimicrobial comprising carvacrol, anethole, a ginger essential oil, and liquorice; and
e) an acceptable carrier.

19. A livestock feed composition comprising the livestock dietary supplement composition of claim 1.

20. A method of feeding livestock comprising administering an effective amount of the livestock feed composition of claim 19, to a livestock animal in need thereof.

21.-40. (canceled)
